SEQUENCE LISTING (10 sequences)

SEQ ID NO: 1 - DNA, 40 bp, Artificial Sequence
  Description of Artificial Sequence Synthetic Primer
gcctaatacgactcactataggacctacggcgtgcagtgc

SEQ ID NO: 2 - DNA, 40 bp, Artificial Sequence
  Description of Artificial Sequence Synthetic Primer
ttgatttaggtgacactatagatggtgcgctcctggacgt

SEQ ID NO: 3 - DNA, 39 bp, Artificial Sequence
  Description of Artificial Sequence Synthetic Primer
gcctaatacgactcactatagacctacggcgtgcagtgc

SEQ ID NO: 4 - DNA, 44 bp, Artificial Sequence
  Description of Artificial Sequence Synthetic Primer
ttttttttttttttttttttttttgatggtgcgctcctggacgt

SEQ ID NO: 5 - DNA, 21 bp, Artificial Sequence
  Description of Combined DNA/RNA Molecule Synthetic siRNA oligo
gcagcacgacuucuucaagtt

SEQ ID NO: 6 - DNA, 21 bp, Artificial Sequence
  Description of Combined DNA/RNA Molecule Synthetic siRNA oligo
cuugaagaagucgugcugctt

SEQ ID NO: 7 - RNA, 21 nt, Unknown Organism
  Description of Unknown Organism GFP mRNA target site sequence
aagcagcacgacuucuucaag

SEQ ID NO: 8 - RNA, 21 nt, Unknown Organism
  Description of Unknown Organism RFP mRNA target site sequence
aagugggagcgcgugaugaac

SEQ ID NO: 9 - DNA, 21 bp, Artificial Sequence
  Description of Combined DNA/RNA Molecule Synthetic siRNA oligo
gugggagcgcgugaugaactt

SEQ ID NO: 10 - DNA, 21 bp, Artificial Sequence
  Description of Combined DNA/RNA Molecule Synthetic siRNA oligo
guucaucacgcgcucccactt

(19) United States
(12) Patent Application Publication
(10) Pub. No.: US 2006/0069050 A1
(43) Pub. Date: Mar. 30, 2006

(54) Methods and compositions for mediating gene silencing

(75) Inventor: Tariq M. Rana, Shrewsbury, MA (US)

Correspondence Address: LAHIVE & COCKFIELD, LLP., 28 STATE STREET, BOSTON, MA, 02109, US

(73) Assignee: UNIVERSITY OF MASSACHUSETTS, Boston, MA (US)

(21) Appl. No.: 11/060,905

(22) Filed: Feb. 17, 2005

Related U.S. Application Data
(60) Provisional application No. 60/545,586, filed on Feb. 17, 2004.

(51) Int. Cl. A61K 48/00; C07H 21/04; C12N 5/08; C12N 15/87

(52) U.S. Cl. 514/44; 435/455; 536/23.2; 435/366

(57) ABSTRACT

The present invention provides methods of conducting RNAi using siRNAs that are sequentially administered as single-stranded oligonucleotides. The siRNAs can be canonical or have non-canonical ends. The compositions and methods of the invention can bypass activation of interferon pathways and yet still efficiently and specifically activate RNAi/gene silencing. In another embodiment, the siRNAs of the invention are modified to allow for the calculation of certain RNAi activities, e.g., RISC activity. The invention also provides methods of using the compositions in research, diagnostic, and therapeutic applications.

### RELATED INFORMATION

The application claims priority to U.S. provisional patent application No. 60/545,586, filed on Feb. 17, 2004, the entire contents of which are hereby incorporated by reference.

The contents of any patents, patent applications, and references cited throughout this specification are hereby incorporated by reference in their entireties.

### STATEMENT AS TO FEDERALLY SPONSORED RESEARCH

Funding for the work described herein was at least in part provided by the federal government under grant numbers AI 41404 and AI 43198, awarded by the United States National Institutes of Health and the National Institute of Allergy and Infectious Diseases.

### BACKGROUND OF THE INVENTION

Double stranded RNA (dsRNA) induces a sequence-specific degradation of homologous mRNA in the cellular process known as RNA interference (RNAi). DsRNA-induced gene silencing has been observed in evolutionarily diverse organisms such as nematodes, flies, plants, fungi, and mammalian cells. Although the entire mechanism of RNAi has not yet been elucidated, several key elements have been identified. RNAi is initiated by an ATP-dependent processive cleavage of dsRNA into 21-23 nucleotide short interfering RNAs (siRNAs) by the DICER endonuclease. The siRNAs are then incorporated into an RNA-induced silencing complex (RISC). This protein and RNA complex is activated by ATP-dependent unwinding of the siRNA duplex. The activated RISC utilizes the antisense strand, also referred to as the guide strand, of the siRNA to recognize and cleave the corresponding mRNA, resulting in decreased expression of the protein encoded by the mRNA.

There recently has been a great deal of interest in the use of RNAi for basic research purposes and for the development of therapeutics to treat, e.g., disorders and/or diseases associated with unwanted or aberrant gene expression, however, siRNA effectiveness at mediating RNAi varies greatly, and can be affected by a number of factors including, but not limited to, the size of the siRNA, the size and nature of any overhangs, and the specificity of the siRNA. Even siRNAs having optimal length, overhangs and specificity, can be ineffective at mediating RNAi.

There is a need for further study of such systems. Moreover, there exists a need for the development of methods and reagents suitable for use in vitro and in vivo, in particular for use in developing human therapeutics.

### SUMMARY OF THE INVENTION

The present invention is based on the surprising discovery that nucleic acids previously thought to be ineffective in RNAi/gene silencing applications because of having non-canonical ends, e.g., having a non-canonical length (i.e., being shorter than 21 nucleotides) or non-canonical overhang (i.e., lacking a 3′dTdT overhang) are as effective as RNAi/gene silencing agents. Accordingly, the invention provides RNAi/gene silencing reagents that bypass the need for 21 nucleotide siRNAs for conducting RNAi.

Moreover, the invention provides for the separate and temporal administration of single-stranded nucleic acids that are as effective as canonical (duplexed and annealed) siRNA agents for carrying out RNAi/gene silencing. The single-stranded nucleic acids administered separately and over time, have the profound advantage of bypassing the interferon response pathway and yet being effective RNAi/gene silencing agents. Because the interferon pathway is triggered by cells exposed to double-stranded nucleic acids, previous RNAi/gene silencing approaches using such agents could not rule out the concomitant activation of this pathway. Accordingly, the invention provides compositions and methods for conducting RNAi/gene silencing both in vitro and in vivo in the absence of an interferon response. This is critical for accurate in vitro screens of gene activities using RNAi and more effective therapeutic applications of RNAi independent of an interferon response.

Still further, the invention provides compositions and methods for revealing the stoichiometry of RNAi/gene silencing machinery. In particular, by administering a titration of double-stranded siRNA nucleic acids having one or more nucleotide modifications, e.g., 2′-O-methylation, against an unmodified siRNA, a calculation of per cell amounts of RNAi activity, e.g., RISC activity, can be determined.

Accordingly, the invention has several advantages which include, but are not limited to, the following:

providing non-canonical RNAi/gene silencing agents equally effective for carrying out RNAi/gene silencing,

providing methods and compositions for carrying out RNAi/gene silencing in the absence of an interferon response by separate and independent administration of an RNAi agent, and

providing methods and compositions for revealing the stoichiometry of RNAi/gene silencing machinery.

Other features and advantages of the invention will be apparent from the following detailed description and claims.

### BRIEF DESCRIPTION OF THE DRAWINGS

FIG. 1 summarizes the efficacy assay used to test various RNAi agents of the invention including the target sequence of the reporter genes (panel A), cell response and fluorescence (panels B and C), and time course data with controls (panel D).

FIG. 2 is a histogram indicating that the sense and antisense strand of an siRNA can be introduced into a cell separately, in either order, and over a time period time of up to 3 days, and still retain RNAi activity.

FIG. 3 shows a panel of siRNA agents comprising non-canonical overhangs as a function of sense strand shortening and/or deletion of the dTdT end. The ends or corresponding panel of antisense strand shortening and/or deletion of the dTdT end (not shown) mirror the panel of molecules shown except that the top strand (sense strand) is canonical or wild type and the alterations are made to the lower antisense strand.

FIG. 4 is a histogram indicating that selected siRNA molecules shown in FIG. 3 and comprising short oligonucleotides are as effective as conventional siRNA agents even though the siRNA agents of the invention comprise a shortened strand and a non-canonical end or overhang. Moreover, the siRNA agents of the invention are effective whether annealed or merely mixed as non-annealed strands (compare “DX” (conventional siRNA agent) with “DX3′d3dT” (annealed but shortened strand with non-canonical overhang) and “Mix(3′dT)” (the foregoing where the strands are mixed, i.e., non-annealed).

FIG. 5 is a histogram showing that a duplexed (i.e., annealed) siRNA when 2′O methylated throughout and titrated against canonical siRNA can reveal the stoichiometry of the RNAi machinery.

FIG. 6 is a graph showing the activity of the modified siRNA at increasing concentrations. These data allow for extrapolating the concentration of RISC in a single mammalian cell as between 0.2 and 2.0 nM but more typically approximately 1.7 nM.

FIG. 7 shows the RNAi activity of sense strand siRNA deletions. Each GFP siRNA construct shown and reporter plasmids were transfected into HeLa cells and RNAi activity was quantified by the dual fluorescence assay 48 h post-transfection. Relative RNAi Activity represents the percentage of GFP knockdown induced by 50 nM of sense strand deletion siRNA relative to the inhibition induced by 50 nM of 19-nt dTdT wild-type siRNA (SS_{1-19dTdT}/AS_{1-19dTdT}; designated 100%).

FIG. 8 shows 16-nt dTdT siRNA targets CDK9 for RNAi-mediated silencing in HeLa cells. Quantitative PCR (qPCR) analysis of CDK9 mRNA knockdown using 1 ug of total RNA from cells transfected with CDK9 19-nt dTdT (SS_{1-19dTdT}/AS_{1-19dTdT}), 16-nt dTdT (SS_{1-16dTdT}/AS_{4-19dTdT}), or 16-nt dTdT (SS_{4-19dTdT}/AS_{1-16dTdT}) siRNA was reverse-transcribed (panel A). CDK9 mRNA levels were quantified using qPCR, were normalized to GAPDH mRNA, and are presented relative to RNA levels in mock-transfected cells. Immunoblot analysis of CDK9 knockdown by 19-nt dTdT and 16-nt dTdT siRNAs is shown in panel B. Cells transfected as shown in panel A were harvested at 48 h post transfection. 120 ug of total protein was analyzed using anti-CDK9 and anti-CycT1 antibodies.

FIG. 9 shows the effects of antisense strand siRNA deletions on RNAi activity. The relative RNAi activity of each GFP siRNA construct shown was evaluated as described in FIG. 7.

FIG. 10 shows a determination of 19-nt dTdT and 16-nt dTdT GFP siRISC* concentration in HeLa cells. HeLa cells were transfected with 19-nt dTdT (SS_{1-19dTdT}/AS_{1-19dTdT}) or 16-nt dTdT (SS_{1-16dTdT}/AS_{4-19dTdT}) GFP siRNA to program siRISC*. A 126-nt ³²P-cap-labeled GFP target RNA was incubated with cell extracts simultaneously with increasing concentrations of 2′ O-methyl RNA oligonucleotides complementary to the GFP target site. The reactions were stopped after a period of 120 min and products were resolved on 6% denaturing polyacrylamide gels. The IC₅₀ analysis was performed using Prizm v.4 software.

FIG. 11 shows 16-nt dTdT siRNA is sufficient for inducing gene knockdown in vivo. The RNAi activity of truncated GFP siRNA ranging from 16-nt dTdT to 13-nt dTdT compared to the RNAi activity of 19-nt dTdT siRNA (panel A). Experiments were performed as described in FIG. 7. Visualizing the siRNA-mediated knockdown in a stable GFP-HeLa cell line is shown in panel B. HeLa cell lines stably expressing EGFP were transfected with truncated GFP siRNA ranging from 16-nt dTdT to 13-nt dTdT. Live images of transfected cells were captured 48 h post-transfection and specific and potent knockdown of GFP expression was detected in cells treated with 19-nt dTdT (SS_{1-19dTdT}/AS_{1-19dTdT}) and 16-nt dTdT (SS_{1-16dTdT}/AS_{4-19dTdT}) siRNAs.

FIG. 12 shows a determination of 19-nt dTdT and 16-nt dTdT GFP siRISC* concentration in HeLa cells. HeLa cells were transfected with 19-nt dTdT (SS_{1-19dTdT}/AS_{1-19dTdT}) or 16-nt dTdT (SS_{1-16dTdT}/AS_{4-19dTdT}) CDK9 siRNA to program siRISC*. A 150-nt ³²P-cap-labeled CDK9 target RNA was incubated with cell extracts simultaneously with increasing concentrations of 2′ O-methyl RNA oligonucleotides complementary to the GFP target site. The reactions were stopped after a period of 120 min and products were resolved on 6% denaturing polyacrylamide gels. The IC₅₀ analysis was performed using Prizm v.4 software.

FIG. 13 shows the in vitro cleavage by 16-nt dTdT and 19-nt dTdT siRISC* (panel A), in vitro cleavage activity of GFP siRISC* programmed with 16-nt dTdT or 19-nt dTdT siRNA (panel B), and in vitro cleavage activity of CDK9 siRISC* programmed with 16-nt dTdT or 19-nt dTdT siRNA. The siRNAs used to program RISC are shown in the schematics and correspond to the numbered gel lanes. Arrowheads in the schematics mark where the target cleavage site is defined by the 5′-end of the antisense strand. The arrow designated ³²P-cap-labeled target points to full-length ³²P-cap-labeled GFP target RNA (panel A) or full-length ³²P-cap-labeled CDK9 target RNA (panel B), respectively. The arrow designated Cleavage product (AS_{1-19dTdT}) and Cleavage product (AS_{4-19dTdT}) point to cleavage products resulting from target RNA cleavage by GFP siRISC* (panel A) or by CDK9 siRISC* (panel B) programmed with 19-nt dTdT and 16-nt dTdT siRNA, respectively.

FIG. 14 shows the RNA-inducing capacity of 16-nt dTdT siRNA over time (panel A). Kinetics of RNAi-mediated knockdown induced by 19-nt dTdT (SS_{1-19dTdT}/AS_{1-19dTdT}) and 16-nt dTdT (SS_{1-16dTdT}/AS_{4-19dTdT}) siRNA in HeLa cells. GFP knockdown is represented by the ratio of normalized GFP/RFP fluorescence and is shown over a 72 h time period (panels B and C). Dose-dependent knockdown of GFP expression by 19-nt dTdT and 16-nt dTdT siRNAs 12 h (B) and 60 h (C) post-transfection.

FIG. 15 shows the target recognition by 16-nt dTdT and 19-nt dTdT siRISC*. In vitro cleavage of GFP RNA by siRISC* co-programmed with varying concentrations of 16-nt dTdT and 19-nt dTdT siRNA is shown in panel A. HeLa cells were co-transfected with different concentrations of 16-nt dTdT or 19-nt dTdT siRNA and harvested at 18 h post transfection. Cell extracts were prepared and incubated with GFP target RNA to evaluate cleavage activity in vitro as described in FIG. 13. The 16-nt dTdT siRISC* and 19-nt dTdT siRISC* compete for target RNA as shown in panel B. The 16-nt dTdT siRNA, perfect match (PF) 19-nt dTdT siRNA or mismatch (MM) 19-nt dTdT siRNA was transfected into HeLa cells, and cell extracts were prepared at 18 h post-transfection. Arrows designated ³²P-cap-labeled target points to fill-length ³²P-cap-labeled GFP target RNA. The arrow designated Cleavage product (AS_{1-19dTdT}) and Cleavage product (AS_{4-19dTdT}) point to cleavage products resulting from target RNA cleavage by GFP siRISC* programmed with 19-nt dTdT and 16-nt dTdT siRNA, respectively.

### DETAILED DESCRIPTION OF THE INVENTION

In order to provide a clear understanding of the specification and claims, the following definitions are conveniently provided below.

Definitions

So that the invention may be more readily understood, certain terms are first defined.

The term “RNA interference” (“RNAi”) or “RNAi activity” refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of a target gene(s).

The phrase “an siRNA having a sequence sufficiently complementary to a target mRNA sequence to direct target-specific RNA interference (RNAi)” refers to a siRNA having sequence sufficient to trigger the destruction of the target mRNA by the RNAi machinery or process.

The term “small interfering RNA” (“siRNA”) (also referred to in the art as “short interfering RNAs”) refers to an RNA (or RNA analog) including strand(s) (e.g., sense and/or antisense strands) comprising between about 10-50 nucleotides (or nucleotide analogs) which is capable of directing or mediating RNA interference.

The term “siRNA duplex” refers to an siRNA having complimentary stands, e.g., a sense strand and antisense strand, wherein the strands are base-paired or annealed (e.g., held together by hydrogen bonds).

The term “non-canonical” siRNA refers to a siRNA having a structure other than that of a classic or canonical siRNA (i.e., a duplex comprising sense and antisense (or guide) strands of about 20-22 nucleotides in length, aligned such that the 3′ ends of the strands extend or overhang the 5′ ends of the complementary strands. Preferably, the non-canonical siRNAs of the invention include an antisense strand of about 19, 20, 21, or 22 nucleotides in length and a shortened or truncated sense strand (e.g., a sense strand of about 10, 11, 12, 13, 14, 15, 16, 17, 18, 19, 20,or21 nucleotides in length). The sense strand can be shortened or truncated at the 5′ end and aligned such that its 3′ end overhangs the 5′ end of the antisense strand (e.g., a 2-3 nucleotide overhang (or more), for example a dTdT overhang). The sense strand can be shortened or truncated at the 3′ end and aligned such that the 3′ end of the antisense strand overhangs its 5′ end. The sense strand can be shortened or truncated at the 3′ end, the 3′ end further comprising 2-3 non-complementary nucleotides (e.g., dTdT), the sense strand being aligned such that the 3′ end of the antisense strand overhangs its 5′ end. The sense strand can be shortened or truncated at both ends, the 3′ end, optionally, further comprising 2-3 non-complementary nucleotides or more (e.g., dTdT). The above-mentioned shortening/truncations are also contemplated for the antisense strand in relation to a sense strand of about 19, 20, 21, or 22 nucleotides in length.

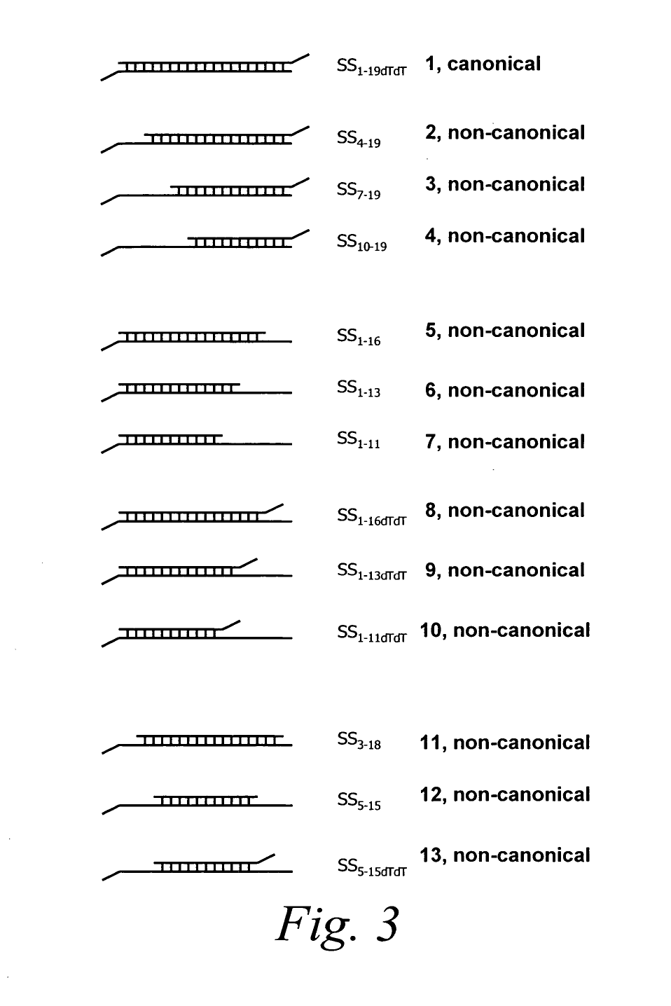

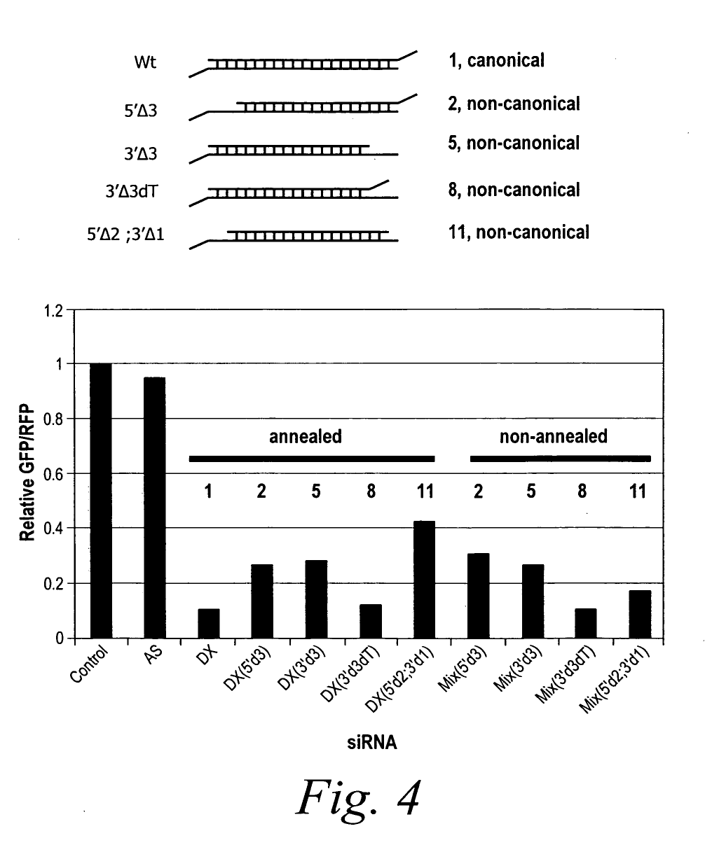

The term “non-canonical siRNA” can also refer to an siRNA having a non-canonical strand length(s) and/or end(s) or overhang(s). A non-canonical strand length is typically less than 21 nucleotides but at least about 10 nucleotides. The term “non-canonical overhang” refers to the atypical end or overhang formed when the mixed, duplexed, or single stranded nucleic acids of the invention are aligned or annealed (in vitro or in vivo). The end(s) or overhang(s) are distinguished from a “canonical” (or wild type) end or overhang of an siRNA in that the end or overhang lacks a 2-nucleotide overhang (e.g., dTdT) and/or one or more nucleotides. Accordingly, non-canonical ends include a 5′ ends with 1, 2, 3, 4, 5, 6, 7, 8, 9, 10 or more nucleotide deletions (or truncations) and/or no dTdT (also referred to as a 5′ non-canonical end) as well as a 3′ end with 1, 2, 3, 4, 5, 6, 7, 8, 9, 10 or more nucleotide deletions and/or no dTdT (also referred to as a 3′ non-canonical end). Exemplary non-canonical siRNAs are shown in FIGS. 3-4.

The term “target gene sequence” refers to a gene sequence encoding a nucleic acid or polypeptide gene product which can be targeted for degradation, e.g., by RNA interference or a RISC-mediated pathway. The target sequenced may be an artificial, recombinant, or naturally occurring sequence. In one embodiment, the sequence encodes a gene product that, when expressed, e.g., at aberrant levels, results in a undesired phenotype, disorder, or disease, in for example, a model organism or human subject.

The phrase “separately and temporally” refers to priming agents, and siRNAs of the invention that exist or are expressed as separate strands, e.g., a sense single-strand and an antisense single strand that are introduced, e.g., to an extract, cell, or organism as a non-annealed mixture or separately, i.e., unmixed, with, preferably, one strand being introduced first followed after a time interval (e.g., several minutes to about 1 hour or more, e.g., 24, 48, or 72 hours), the second strand.

The term “priming agent” refers to a compound, typically a nucleic acid, e.g., a oligonucleotide or single-stranded nucleic acid, mixture or annealed nucleic acid, siRNA, shRNA, non-canonical siRNAs, or even non-sequence specific nucleic acids, which can be used to enhance or “prime”, “program”, “activate”, or “trigger” an RNAi pathway, e.g., RISC activity, in a cell extract, cell, or organism. Typically, the priming agent is introduced or expressed in the cell using art recognized techniques.

The term “RISC” or “RNA induced silencing complex” refers to the nucleic acid and polypeptide components, e.g., Dicer, R2D2, and the Argonaute family of polypeptides, that interact to recognize target gene sequences, e.g., RNA molecules for targeted destruction or silencing. This activity is also referred to as “RISC activity” or “RNA induced silencing complex activity”.

The term “high level of activated RISC” refers to a level of RISC activity, e.g., as measured by target gene degradation, which is sufficiently elevated or above what is usual for a comparable/control extract, cell, or organism. For example, in mammalian cells, e.g., HeLa cells, the high level of RISC activity is calculated to be about 0.2 to about 1.9 nM or more for a single cell. Typically, the high level of activated RISC is achieved by priming a cell, cell extract, or organism by exposing the cell, cell extract, or organism to a priming agent as described herein. Changes in primed RISC activity as compared to a control result in a fold increase of 1.5, 2, 3, 4, 5, 10, 15, 20, or more.

The term “nucleic acid” and “single-stranded nucleic acid” refers to RNA or RNA molecules as well as DNA molecules. The term RNA refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively), or multi-stranded (e.g., double stranded, i.e., dsRNA and dsDNA, respectively), i.e., duplexed or annealed.

The term “modified nucleotide” or “modified nucleic acid(s)” refers to a non-standard nucleotide or nucleic acid, including non-naturally occurring ribonucleotides or deoxyribonucleotides. Preferred nucleotide analogs or nucleic acids are modified at any position so as to alter certain chemical properties, e.g., increase stability of the nucleotide or nucleic acid yet retain its ability to perform its intended function, e.g., have priming and/or RNAi activity. Examples include methylation at one or more bases, e.g., O-methylation, preferably 2′ O methylation (2′-O-Me), dyes which can be linked to the nucleic acid to provide for visual detection of the nucleic acid, and biotin moieties which can be used to purify the nucleic acid to which it is attached as well as any associated components bound to the biotinylated nucleic acid. Other examples of modified nucleotides/nucleic acids are described in Herdewijn, Antisense Nucleic Acid Drug Dev., August 2000 10(4):297-3 10; U.S. Pat. Nos. 5,858,988; 6,291,438; Eckstein, Antisense Nucleic Acid Drug Dev. April 2000 10(2): 117-2 1; Rusckowski et al. Antisense Nucleic Acid Drug Dev. October 2000 10(5):333-45; Stein, Antisense Nucleic Acid Drug Dev. October 2001 11(5): 317-25; Vorobjev et al. Antisense Nucleic Acid Drug Dev. April 2001 11(2):77-85; and U.S. Pat. No. 5,684,143.

A gene “involved” in a disorder includes a gene, the normal or aberrant expression or function of which effects or causes a disease or disorder or at least one symptom of the disease or disorder

The phrase “examining the function of a gene in a cell or organism” refers to examining or studying the expression, activity, function or phenotype arising therefrom. Various methodologies of the invention include a step that involves comparing a value, level, feature, characteristic, property, etc. to a “suitable control”, referred to interchangeably herein as an “appropriate control”.

A “suitable control” or “appropriate control” refers to any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a RISC level of activity or amount, target gene level or target gene degradation level, a transcription rate, mRNA level, translation rate, protein level, biological activity, cellular characteristic or property, genotype, phenotype, etc. can be determined prior to introducing a nucleic acid of the invention into a cell, cell extract, or organism.

The term “cell” refers to any eukaryotic cell which exhibits RNAi activity and includes, e.g., animal cells (e.g., mammalian cells, e.g., human or murine cells), plant cells, and yeast. The term includes cell lines, e.g., mammalian cell lines such as HeLa cells as well as embryonic cells, e.g., embryonic stem cells and collections of cells in the form of, e.g., a tissue.

The term “cell extract” refers to a lysate or acellular preparation of a cell as defined above and can be a crude extract or partially purified as well as comprise additional agents such as recombinant polypeptides, nucleic acids, and/or buffers or stabilizers.

The term “organism” refers to multicellular organisms such as, e.g., C. elegans, Drosophila, mouse, and human.

The term “vector” refers to a nucleic acid molecule (either DNA or RNA) capable of conferring the expression of a gene product when introduced into a host cell or host cell extract. In one embodiment, the vector allows for temporal or conditional expression of one or more nucleic acids of the invention, e.g., a priming agent, single strand, siRNA, non-canonical siRNA, or shRNA. The vector may be episomal or chromosomally (e.g., transgenically) integrated into the host cell genome.

### Detailed Description

Overview

The invention features small interfering RNA (siRNA), comprising a sense strand and an antisense strand, the antisense strand having a sequence sufficiently complementary to a target gene sequence to direct target-specific RNA interference (RNAi), wherein the strands, when aligned, form at least one non-canonical overhang or end. The non-canonical siRNAs of the invention include an siRNA having a first non-canonical end; an siRNA having a second non-canonical end; an siRNA having a first and a second non-canonical end; an siRNA wherein the sense strand can be shortened or truncated at the 5′ end and aligned such that its 3′ end overhangs the 5′ end of the antisense strand; an siRNA wherein the sense strand can be shortened or truncated at the 3′ end and aligned such that the 3′ end of the antisense strand overhangs its 5′ end; an siRNA wherein the sense strand can be shortened or truncated at the 3′ end, the 3′ end further comprising 2-3 non-complementary nucleotides, the sense strand being aligned such that the 3′ end of the antisense strand overhangs its 5′ end; or an siRNA wherein the sense strand can be shortened or truncated at both ends, the 3′ end, optionally, further comprising 2-3 non-complementary nucleotides (or more). Exemplary non-canonical siRNAs are shown in FIGS. 3-4, 7, 9, 11, and 13.

The invention also provides small interfering RNA (siRNA), comprising a sense strand and an antisense strand, the antisense strand having a sequence sufficiently complementary to a target gene sequence to direct target-specific RNA interference (RNAi), wherein the sense strand and antisense strand are separately and temporally exposed to a cell, cell lysate, or organism. The separate administration of each strand where there is a time interval between the introduction of each strand, can be performed with canonical or non-canonical siRNA. Time intervals of several minutes to about an hour or more, e.g., 12, 24, 48, and 72 hours or more, are encompassed by the invention.

The first strand administered can also function as a priming agent and enhance the level of RISC or RNAi responsiveness of the cell, cell extract, or organism such that the second strand, when introduced, has improved effect.

Accordingly, siRNAs of the above aspects can comprise a sense strand of about 21 nucleotides (e.g., 19, 20, 21, or 22 nucleotides) and corresponding antisense strand of at least 10, 11, 12, 13, 14, 15, 16, 17, 18 to 19 nucleotides or an antisense strand of about 21 nucleotides (e.g., 19, 20, 21, or 22 nucleotides) and corresponding sense strand of at least 10, 11, 12, 13, 14, 15, 16, 17, 18 to 19 nucleotides. Importantly, when each strand is administered separately, the siRNA directs target specific interference and bypasses an interferon response pathway. siRNAs comprising a sense strand of 14, 15, or 16 nucleotides are particularly effective (see Examples 4-7).

The gene silencing agents of the invention can be in a pharmaceutically acceptable carrier or liposome. The gene silencing agents of the invention may also be expressed in a cell and therefore encoded in a vector, preferably a vector capable of conditional expression and/or tissue specific expression. The tet operator and operon is a preferred conditional expression system.

The invention also provides cells having the above gene silencing agents, for example, as expressed from a vector, maintained episomally or chromosomally integrated (e.g. transgenically) into the genome of the cell. Accordingly, organisms, for example transgenic organisms, may be derived or comprise such a cell, and include non-human transgenic organisms such as a transgenic mouse.

In another aspect, the invention provides a method of activating target-specific RNA interference (RNAi) in a cell by introducing into the cell a small interfering RNA (siRNA), comprising a sense strand and an antisense strand, the antisense strand having a sequence sufficiently complementary to a target gene sequence to direct target-specific RNA interference (RNAi), wherein the strands, when aligned, form at least one non-canonical overhang. The siRNA is introduced in an amount sufficient for degradation of target mRNA to occur, thereby activating target-specific RNAi in the cell. In a preferred embodiment, the sense and antisense strand are introduced separately, and preferably, over a time interval of about 1 hour or more.

In one embodiment, the RNAi agents, e.g., siRNAs, are introduced into the cell by contacting the cell, in particular, with a composition comprising the siRNA and a lipophilic carrier.

In another embodiment, the siRNA is introduced into the cell by transfecting or infecting the cell with a vector comprising nucleic acid sequences capable of producing the siRNA when transcribed in the cell.

In still another embodiment, the siRNA is introduced into the cell by injecting into the cell a vector comprising nucleic acid sequences capable of producing the siRNA when transcribed in the cell. The vector may further comprise transgene nucleic acid sequences. The invention also encompasses cells made according to the foregoing, in particular, cells of mammalian origin, e.g., embryonic stem cells, or murine or human cells, including human cell lines such as HeLa cells, as well as non-human organisms.

In a preferred embodiment of the method, the target mRNA specifies the amino acid sequence of a protein involved or predicted to be involved in a human disease or disorder.

In another aspect, the invention provides a method of activating target-specific RNA interference (RNAi) in an organism by administering to the organism an siRNA as described above, the siRNA being administered in an amount sufficient for degradation of the target mRNA to occur, thereby activating target-specific RNAi in the organism, e.g., a mammalian organism, including, e.g., a human subject.

In one embodiment, the target mRNA specifies the amino acid sequence of a protein involved or predicted to be involved in a human disease or disorder.

Accordingly, the invention also provides a method of treating a disease or disorder associated with the activity of a protein specified by a target mRNA in a subject by administering to the subject an siRNA as described above in an amount sufficient for degradation of the target mRNA to occur, thereby treating the disease or disorder associated with the protein.

Still further, the invention provides methods for deriving information about the function of a gene in a cell or organism by introducing into the cell or organism an siRNA as described above and maintaining the cell or organism under conditions such that target-specific RNAi can occur, determining a characteristic or property of the cell or organism, and comparing the characteristic or property to a suitable control, the comparison yielding information about the function of the gene.

In addition, the invention provides a method of validating a candidate protein as a suitable target for drug discovery by introducing into a cell or organism an siRNA as described above and maintaining the cell or organism under conditions such that target-specific RNAi can occur, determining a characteristic or property of the cell or organism, and comparing the characteristic or property to a suitable control, the comparison yielding information about whether the candidate protein is a suitable target for drug discovery.

In another aspect, the invention provides a kit comprising reagents for activating target-specific RNA interference (RNAi) in a cell or organism, the kit containing an siRNA as described above and instructions for use.

Further details for carrying out various aspects of the invention are provided in the following subsections below.

1. Non-Canonical RNAi Agents, Non-Canonical siRNAs

The present invention features nucleic acids such as “small interfering RNA molecules” (“siRNA molecules” or “siRNA” but also single and double stranded shRNAs) which can be used as gene silencing agents but also as priming agents for enhancing the RISC activity of a cell. Typically, an siRNA molecule of the invention is a duplex consisting of a sense strand and complementary antisense strand, the antisense strand having sufficient complementarity to a target mRNA to mediate RNAi, wherein the molecule is either administered as separate strands (in which case the first strand can serve as a priming agent), as a non-canonical strand(s), or as a non-canonical duplex (either annealed or non-annealed).

siRNAs can be from about 10-50 or more nucleotides, i.e., each strand comprises 10-50 nucleotides (or nucleotide analogs). More preferably, the siRNA molecule has a length from about 15-45 nucleotides. Even more preferably, the siRNA molecule has a length from about 18-25 nucleotides. The siRNA molecules of the invention further have a sequence that is “sufficiently complementary” to a target mRNA sequence to direct target-specific RNA interference (RNAi), as defined herein, i.e., the siRNA has a sequence sufficient to trigger the destruction of the target mRNA by the RNAi machinery or process. Most preferably, the siRNA are non-canonical or administered as separate strands.

2. Producing RNAi and Non-Canonical RNAi Agents

Nucleic acid agents, e.g., RNAi agents, more particularly, non-canonical RNAi agents such as siRNAs, can be produced enzymatically or by partial/total organic synthesis. In one embodiment, an RNAi agent is prepared chemically. Methods of synthesizing RNA molecules are known in the art, in particular, the chemical synthesis methods as de scribed in Verma and Eckstein (1998) Annul Rev. Biochem. 67:99-134. In another embodiment, the nucleic acids are produced enzymatically, e.g., by enzymatic transcription from synthetic DNA templates or from DNA plasmids isolated from recombinant bacteria. Typically, phage RNA polymerases are used such as T7, T3 or SP6 RNA polymerase (Milligan and Uhlenbeck (1989) Methods Enzymol. 180:51-62). In one embodiment, the siRNAs are synthesized either in vivo, in situ, or in vitro. Endogenous RNA polymerase of the cell may mediate transcription in vivo or in situ, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the siRNA. Inhibition may be targeted by specific transcription in an organ, tissue, or cell type; stimulation of an environmental condition (e.g., infection, stress, temperature, chemical inducers); and/or engineering transcription at a developmental stage or age or by conditional expression from a vector or transgene having an inducible promoter or operon. A transgenic organism that expresses a nucleic acid priming agent RNA from a recombinant construct may be produced by introducing the construct into a zygote, an embryonic stem cell, or another multipotent cell derived from the appropriate organism.

3. Modified RNAi Agents

The invention also features small interfering RNAs (siRNAs) that include a sense strand and an antisense strand, wherein the antisense strand has a sequence sufficiently complementary to a target mRNA sequence to direct target-specific RNA interference (RNAi) and wherein the sense strand and/or antisense strand is modified by the substitution of modified nucleotides, such that in vivo stability is enhanced as compared to a corresponding unmodified siRNA. For example, the RNAi agent may be methylated, e.g., 2′O-methylated at one of more bases. Certain modifications confer useful properties to siRNA. For example, increased stability compared to an unmodified siRNA or a label that can be used, e.g., to trace the siRNA, to purify an siRNA, or to purify the siRNA and cellular components with which it is associated. For example, such modifications may be used to stabilize the first (priming) strand for enhancing RISC activity/RNAi responsiveness in a cell (or cell extract or organism) and improve its intracellular half-life for subsequent receipt of the second strand wherein RNAi/gene silencing can now progress. Certain modifications can also increase the uptake of the siRNA by a cell. For example, functional groups such as biotin are useful for affinity purification of proteins and molecular complexes involved in the RNAi mechanism. The invention also includes methods of testing modified siRNAs for retention of the ability to act as an siRNA (e.g., in RNAi) and methods of using siRNA derivatives, e.g., in order to purify or identify RISC components (see, e.g., PCT/US03/36551; PCT/US03/24595; and PCT/US03/30480).

Modifications have the added feature of enhancing properties such as cellular uptake of the siRNAs and/or stability of the siRNAs. Preferred modifications are made at the 2′ carbon of the sugar moiety of nucleotides within the siRNA. Also preferred are certain backbone modifications, as described herein. Also preferred are chemical modifications that stabilize interactions between base pairs, as described herein. Combinations of substitution are also featured. Preferred modifications maintain the structural integrity of the antisense siRNA-target mRNA duplex.

The present invention features modified siRNAs. siRNA modifications are designed such that properties important for in vivo applications, in particular, human therapeutic applications, are improved without compromising the RNAi activity of the siRNA molecules e.g., modifications to increase resistance of the siRNA molecules to nucleases. Modified siRNA molecules of the invention comprise a sense strand and an antisense strand, wherein the sense strand or antisense strand is modified by the substitution of at least one nucleotide with a modified nucleotide, such that, for example, in vivo stability is enhanced as compared to a corresponding unmodified siRNA, or such that the target efficiency is enhanced compared to a corresponding unmodified siRNA. Such modifications are also useful to improve uptake of the siRNA by a cell. Preferred modified nucleotides do not effect the ability of the antisense strand to adopt A-form helix conformation when base-pairing with the target mRNA sequence, e.g., an A-form helix conformation comprising a normal major groove when base-pairing with the target mRNA sequence.

Modified siRNA molecules of the invention (i.e., duplex siRNA molecules) can be modified at the 5′ end, 3′ end, 5′ and 3′ end, and/or at internal residues, or any combination thereof. Internal siRNA modifications can be, for example, sugar modifications, nucleobase modifications, backbone modifications, and can contain mismatches, bulges, or crosslinks. Also preferred are 3′ end, 5′ end, or 3′ and 5′ and/or internal modifications, wherein the modifications are, for example, cross linkers, heterofunctional cross linkers, dendrimer, nano-particle, peptides, organic compounds (e.g., fluorescent dyes), and/or photocleavable compounds.

In one embodiment, the siRNA molecule of the invention comprises one or more (e.g., about 1, 2, 3, 4, 5, 6, 7, 8, 9, 10, or more) end modifications. Modification at the 5′ end is preferred in the sense strand, and comprises, for example, a 5′-propylamine group. Modifications to the 3′ OH terminus are in the sense strand, antisense strand, or in the sense and antisense strands. A 3′ end modification comprises, for example, 3′-puromycin, 3′-biotin and the like.

In another embodiment, the siRNA molecule of the invention comprises one or more (e.g., about 1, 2, 3, 4, 5, 6, 7, 8, 9, 10, or more) crosslinks, e.g., a crosslink wherein the sense strand is crosslinked to the antisense strand of the siRNA duplex. Crosslinkers useful in the invention are those commonly known in the art, e.g., psoralen, mitomycin C, cisplatin, chloroethylnitrosoureas and the like. A preferred crosslink of the invention is a psoralen crosslink. Preferably, the crosslink is present downstream of the cleavage site referencing the antisense strand, and more preferably, the crosslink is present at the 5′ end of the sense strand.

In another embodiment, the siRNA molecule of the invention comprises one or more (e.g., about 1, 2, 3, 4, 5, 6, 7, 8, 9, 10, or more) sugar-modified nucleotides. Sugar-modifed nucleotides useful in the invention include, but are not limited to: 2′-fluoro modified ribonucleotide, 2′-OMe modified ribonucleotide, 2′-deoxy ribonucleotide, 2′-amino modified ribonucleotide and 2′-thio modified ribonucleotide. The sugar-modified nucleotide can be, for example, 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine or 2′-amino-butyryl-pyrene-uridine. A preferred sugar-modified nucleotide is a 2′-deoxy ribonucleotide. Preferably, the 2′-deoxy ribonucleotide is present within the sense strand and, for example, can be upstream of the cleavage site referencing the antisense strand or downstream of the cleavage site referencing the antisense strand. A preferred sugar-modified nucleotide is a 2′-fluoro modified ribonucleotide. Preferably, the 2′-fluoro ribonucleotides are in the sense and antisense strands. More preferably, the 2′-fluoro ribonucleotides are every uridine and cytidine.

In another embodiment, the siRNA molecule of the invention comprises one or more (e.g., about 1, 2, 3, 4, 5, 6, 7, 8, 9, 10, or more) nucleobase-modified nucleotides. Nucleobase-modified nucleotides useful in the invention include, but are not limited to: 5-bromo-uridine, 5-iodo-uridine, 5-methyl-cytidine, ribo-thymidine, 2-aminopurine, 5-fluoro-cytidine, and 5-fluoro-uridine, 2,6-diaminopurine, 4-thio-uridine; and 5-amino-allyl-uridine and the like.

In another embodiment, the siRNA molecule of the invention comprises one or more (e.g., about 1, 2, 3, 4, 5, 6, 7, 8, 9, 10, or more) backbone-modified nucleotides, for example, a backbone-modified nucleotide containing a phosphorothioate group. The backbone-modified nucleotide is within the sense strand, antisense strand, or preferably within the sense and antisense strands.

In another embodiment, the siRNA molecule of the invention comprises a sequence wherein the antisense strand and target mRNA sequences comprise one or more (e.g., about 1, 2, 3, 4, 5, 6, 7, 8, 9, 10, or more) mismatches. Preferably, the mismatch is downstream of the cleavage site referencing the antisense strand. More preferably, the mismatch is present within 1-6 nucleotides from the 3′ end of the antisense strand. In another embodiment, the siRNA molecule of the invention comprises a bulge, e.g., one or more (e.g., about 1, 2, 3, 4, 5, 6, 7, 8, 9, 10, or more) unpaired bases in the duplex siRNA. Preferably, the bulge is in the sense strand.

In another embodiment, the siRNA molecule of the invention comprises any combination of two or more (e.g., about 2, 3, 4, 5, 6, 7, 8, 9, 10, or more) siRNA modifications as described herein. For example, a siRNA molecule can comprise a combination of two sugar-modified nucleotides, wherein the sugar-modified nucleotides are 2′-fluoro modified ribonucleotides, e.g., 2′-fluoro uridine or 2′-fluoro cytidine, and 2′-deoxy ribonucleotides, e.g., 2′-deoxy adenosine or 2′-deoxy guanosine. Preferably, the 2′-deoxy ribonucleotides are in the antisense strand, and, for example, can be upstream of the cleavage site referencing the antisense strand or downstream of the cleavage site referencing the antisense strand. Preferably, the 2′-fluoro ribonucleotides are in the sense and antisense strands. More preferably, the 2′-fluoro ribonucleotides are every uridine and cytidine.

The invention is also related to the discovery that certain characteristics of siRNA are necessary for activity and that modifications can be made to an siRNA to alter physicochemical characteristics such as stability in a cell and the ability of an siRNA to be taken up by a cell. Accordingly, the invention includes siRNA derivatives; siRNAs that have been chemically modified and retain activity in RNA interference (RNAi). The invention also includes a dual fluorescence reporter assay (DFRA) that is useful for testing the activity of siRNAs and siRNA derivatives.

Accordingly, the invention includes an siRNA derivative that includes an siRNA having two complementary strands of nucleic acid, such that the two strands are crosslinked, a 3′ OH terminus of one of the strands is modified, or the two strands are crosslinked and modified at the 3′OH terminus. The siRNA derivative can contain a single crosslink (e.g., a psoralen crosslink). In some embodiments, the siRNA derivative has a biotin at a 3′ terminus (e.g., a photocleavable biotin ), a peptide (e.g., a Tat peptide), a nanoparticle, a peptidomimetic, organic compounds (e.g., a dye such as a fluorescent dye), or dendrimer.

4. Selecting a Gene Target

In one embodiment, the target gene sequence or mRNA of the invention encodes the amino acid sequence of a cellular protein, e.g., a protein involved in cell growth or suppression, e.g., a nuclear, cytoplasmic, transmembrane, membrane-associated protein, or cellular ligand. In another embodiment, the target mRNA of the invention specifies the amino acid sequence of an extracellular protein (e.g., an extracellular matrix protein or secreted protein). Typical classes of proteins are developmental proteins, cancer gene such as oncogenes, tumor suppressor genes, and enzymatic proteins, such as topoisomerases, kinases, and telomerases.

In a preferred aspect of the invention, the target mRNA molecule of the invention specifies the amino acid sequence of a protein associated with a pathological condition. By modulating the expression of the foregoing proteins, valuable information regarding the function of such proteins and therapeutic benefits which may be obtained from such modulation can be obtained.

5. Determining Gene Target Sequence Identity

The target RNA cleavage reaction guided by siRNAs (e.g., by siRNAs) is highly sequence specific. In general, siRNA containing a nucleotide sequences identical to a portion of the target gene are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Moreover, not all positions of a siRNA contribute equally to target recognition. Mismatches in the center of the siRNA are most critical and essentially abolish target RNA cleavage. Mismatches upstream of the center or upstream of the cleavage site referencing the antisense strand are tolerated but significantly reduce target RNA cleavage. Mismatches downstream of the center or cleavage site referencing the antisense strand, preferably located near the 3′ end of the antisense strand, e.g. 1, 2, 3, 4, 5 or 6 nucleotides from the 3′ end of the antisense strand, are tolerated and reduce target RNA cleavage only slightly.

Sequence identity may determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

Greater than 90% sequence identity, e.g., 91%, 92%, 93%, 94%, 95%, 96%, 97%, 98%, 99% or even 100% sequence identity, between the siRNA and the portion of the target gene is preferred. Alternatively, the siRNA may be defined functionally as a nucleotide sequence (or oligonucleotide sequence) that is capable of hybridizing with a portion of the target gene transcript. Examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

6. Efficacy Assays

The invention features methods of assaying the ability of a compound of the invention (e.g., a siRNA, candidate RNAi derivative, modified siRNA, etc.) to modulate (e.g., inhibit) expression of a target RNA using a dual fluorescence system. The assay may be used to determine the amount of improved RISC activity after priming the cell. Other assay systems known in the art that measure the efficacy of an siRNA can be modified as described herein to evaluate whether a modified siRNA is also a priming agent.

A compound of the invention (e.g., a priming agent, a siRNA, candidate priming agent, candidate RNAi derivative, modified siRNA, etc.) can be tested for its ability to improve a cell or cell extract RISC activity and responsiveness in inhibiting expression of a targeted gene. For example, candidate RNAi derivatives that can inhibit such expression are identified as siRNA derivatives. Any system in which RNAi activity can be detected can be used to test the activity of a compound of the invention (e.g., a siRNA, candidate priming agent, candidate RNAi derivative, modified siRNA, etc.). In general, a system in which RNAi activity can be detected is incubated in the presence and absence of a compound of the invention (e.g., a siRNA, candidate priming agent, candidate RNAi derivative, modified siRNA, etc.).

The invention includes a dual fluorescence reporter gene assay (DFRG assay) that can be used to test a compound of the invention (e.g., a priming agent, candidate priming agent, a siRNA, non-canonical siRNA, candidate RNAi derivative, modified siRNA, etc.). The DFRG assay can also be used, for example, to test the ability of these and other types of compounds to inhibit expression of a targeted gene. Technical details of the assay are provided in PCT/US03/30480 which is incorporated by reference in its entirety.

7. Methods of Introducing RNAi Agents into Cells

Physical methods of introducing nucleic acids include injection of a solution containing the nucleic acid, bombardment by particles covered by the nucleic acid, soaking the cell or organism in a solution of the nucleic acid, or electroporation of cell membranes in the presence of the nucleic acid. A viral construct packaged into a viral particle would accomplish both efficient introduction of an expression construct into the cell and transcription of nucleic acid encoded by the expression construct. Other methods known in the art for introducing nucleic acids to cells may be used, such as lipid-mediated carrier transport, chemical-mediated transport, such as calcium phosphate, and the like. Thus the nucleic acid may be introduced along with components that perform one or more of the following activities: enhance nucleic acid uptake by the cell, inhibit annealing of single strands, stabilize the single strands, or other-wise increase inhibition of the target gene.

Nucleic acid may be directly introduced into the cell (i.e., intracellularly); or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

The cell with the target gene may be derived from or contained in any organism. The organism may a plant, animal, protozoan, bacterium, virus, or fungus. The plant may be a monocot, dicot or gymnosperm; the animal may be a vertebrate or invertebrate. Preferred microbes are those used in agriculture or by industry, and those that are pathogenic for plants or animals.

Alternatively, vectors, e.g., transgenes encoding a priming agent/siRNA of the invention can be engineered into a host cell or transgenic animal using art recognized techniques.

8. Cells/Vectors/and Uses Therefore

A further preferred use for the agents of the present invention (or vectors or transgenes encoding same) is a functional analysis to be carried out in eukaryotic cells, or eukaryotic non-human organisms, preferably mammalian cells or organisms and most preferably human cells, e.g. cell lines such as HeLa or 293 or rodents, e.g. rats and mice. By administering a suitable priming agent/RNAi agent which is sufficiently complementary to a target mRNA sequence to direct target-specific RNA interference, a specific knockout or knockdown phenotype can be obtained in a target cell, e.g. in cell culture or in a target organism.

Thus, a further subject matter of the invention is a eukaryotic cell or a eukaryotic non-human organism exhibiting a target gene-specific knockout or knockdown phenotype comprising a fully or at least partially deficient expression of at least one endogenous target gene wherein said cell or organism is transfected with at least one vector comprising DNA encoding an RNAi agent capable of inhibiting the expression of the target gene. It should be noted that the present invention allows a target-specific knockout or knockdown of several different endogenous genes due to the specificity of the RNAi agent.

Gene-specific knockout or knockdown phenotypes of cells or non-human organisms, particularly of human cells or non-human mammals may be used in analytic to procedures, e.g. in the functional and/or phenotypical analysis of complex physiological processes such as analysis of gene expression profiles and/or proteomes. Preferably the analysis is carried out by high throughput methods using oligonucleotide based chips.

9. Screening Assays

The methods of the invention are also suitable for use in methods to identify and/or characterize RNAi agents, pharmacological agents, e.g. identifying new RNAi agents, pharmacological agents from a collection of test substances and/or characterizing mechanisms of action and/or side effects of known RNAi agents or pharmacological agents.

Thus, the present invention also relates to a system, for example, a high throughput system (HTS), for identifying and/or characterizing pharmacological agents acting on at least one target protein comprising: a eukaryotic cell, cell extract, or a eukaryotic non-human organism primed or capable of being primed and expressing at least one endogenous target gene coding for a target protein, at least one priming/RNAi agent molecule capable of enhancing RISC activity or RNA responsiveness and inhibiting the expression of at least one endogenous target gene, and a test substance or a collection of test substances wherein the properties of the test substance or collection of test substances are to be identified and/or characterized.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad Sci. 87:6378-6382); (Felici (1991) J. Mol Biol. 222:301-310); (Ladner supra.)).

In a preferred embodiment, the library is a natural product library, e.g., a library produced by a bacterial, fungal, or yeast culture. In another preferred embodiment, the library is a synthetic compound library.

This invention is further illustrated by the following examples which should not be construed as limiting.

10. Transgenic Organisms

Engineered priming/RNAi agents of the invention can be expressed in transgenic animals. These animals represent a model system for the study of disorders that are caused by, or exacerbated by, overexpression or underexpression (as compared to wildtype or normal) of nucleic acids (and their encoded polypeptides) targeted for destruction by the RNAi agents, e.g., siRNAs and shRNAs, and for the development of therapeutic agents that modulate the expression or activity of nucleic acids or polypeptides targeted for destruction.

Transgenic animals can be farm animals (pigs, goats, sheep, cows, horses, rabbits, and the like), rodents (such as rats, guinea pigs, and mice), non-human primates (for example, baboons, monkeys, and chimpanzees), and domestic animals (for example, dogs and cats). Invertebrates such as Caenorhabditis elegans or Drosophila can be used as well as non-mammalian vertebrates such as fish (e.g., zebrafish) or birds (e.g., chickens).

Engineered RNA precursors with stems of 18 to 30 nucleotides in length are preferred for use in mammals, such as mice. A transgenic founder animal can be identified based upon the presence of a transgene that encodes the new RNA precursors in its genome, and/or expression of the transgene in tissues or cells of the animals, for example, using PCR or Northern analysis. Expression is confirmed by a decrease in the expression (RNA or protein) of the target sequence.

Methods for generating transgenic animals include introducing the transgene into the germ line of the animal. One method is by microinjection of a gene construct into the pronucleus of an early stage embryo (e.g., before the four-cell stage; Wagner et al., 1981, Proc. Natl. Acad. Sci. USA 78:5016; Brinster et al., 1985, Proc. Natl. Acad. Sci. USA 82:4438). Alternatively, the transgene can be introduced into the pronucleus by retroviral infection. A detailed procedure for producing such transgenic mice has been described (see e.g., Hogan et al., Manipulating the Mouse Embryo. Cold Spring Harbor Laboratory, Cold Spring Harbor, N.Y. (1986); U.S. Pat. No. 5,175,383 (1992)). This procedure has also been adapted for other animal species (e.g., Hammer et al., 1985, Nature 315:680; Murray et al., 1989, Reprod. Fert. Devl. 1:147; Pursel et al., 1987, Vet. Immunol. Histopath. 17:303; Rexroad et al., 1990, J. Reprod. Fert. 41 (suppl): 1 19; Rexroad et al., 1989, Molec. Reprod. Devl. 1:164; Simons et al., 1988, BioTechnology 6:179; Vize et al., 1988, J. Cell. Sci. 90:295; and Wagner, 1989, J. Cell. Biochem. 13B (suppl): 164). Clones of the non-human transgenic animals described herein can be produced according to the methods described in Wilmut et al. ((1997) Nature, 385:810-813) and PCT publication Nos. WO 97/07668 and WO 97/07669.

11. Methods of Treatment

The present invention provides for both prophylactic and therapeutic methods of treating a subject at risk of (or susceptible to) a disorder or having a disorder associated with aberrant or unwanted target gene expression or activity. In one embodiment, the subject is primed with a priming agent, and then administered an siRNA for suppressing the expression of an the undesired gene product. It is understood that “treatment” or “treating” as used herein, is defined as the application or administration of a therapeutic agent (e.g., a RNAi agent or vector or transgene encoding same) to a patient, or application or administration of a therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

12. Prophylactic Methods

In another aspect, the invention provides a method for preventing in a subject, a disease or condition associated with an aberrant or unwanted target gene expression or activity, by administering to the subject a therapeutic agent (e.g., a RNAi agent or vector or transgene encoding same). If appropriate, subjects are first treated with a priming agent so as to be more responsive to the subsequent RNAi therapy. Subjects at risk for a disease which is caused or contributed to by aberrant or unwanted target gene expression or activity can be identified by, for example, any or a combination of diagnostic or prognostic assays as described herein. Administration of a prophylactic agent can occur prior to the manifestation of symptoms characteristic of the target gene aberrancy, such that a disease or disorder is prevented or, alternatively, delayed in its progression. Depending on the type of target gene aberrancy, for example, a target gene, target gene agonist or target gene antagonist agent can be used for treating the subject. The appropriate agent can be determined based on screening assays described herein.

13. Therapeutic Methods

In yet another aspect, the invention pertains to methods of modulating target gene expression, protein expression or activity for therapeutic purposes. Accordingly, in an exemplary embodiment, the modulatory method of the invention involves contacting a cell capable of expressing target gene with a therapeutic agent (e.g., a priming agent, RNAi agent or vector or transgene encoding same) that is specific for the target gene or protein (e.g., is specific for the mRNA encoded by said gene or specifying the amino acid sequence of said protein) such that expression or one or more of the activities of target protein is modulated. These modulatory methods can be performed in vitro (e.g., by culturing the cell with the agent), in vivo (e.g., by administering the agent to a subject), or ex vivo. Typically, subjects are first treated with a priming agent so as to be more responsive to the subsequent RNAi therapy. As such, the present invention provides methods of treating an individual afflicted with a disease or disorder characterized by aberrant or unwanted expression or activity of a target gene polypeptide or nucleic acid molecule. Inhibition of target gene activity is desirable in situations in which target gene is abnormally unregulated and/or in which decreased target gene activity is likely to have a beneficial effect.

14. Pharmacogenomics

The therapeutic agents (e.g., a RNAi agent or vector or transgene encoding same) of the invention can be administered to individuals to treat (prophylactically or therapeutically) disorders associated with aberrant or unwanted target gene activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a therapeutic agent as well as tailoring the dosage and/or therapeutic regimen of treatment with a therapeutic agent.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266

15. Pharmaceutical Compositions

The invention pertains to uses of the above-described agents for therapeutic treatments as described infra. Accordingly, the modulators of the present invention can be incorporated into pharmaceutical compositions suitable for administration. Such compositions typically comprise the nucleic acid molecule, e.g., priming agent, and together or separately, an RNAi agent, e.g., an siRNA agent for carrying out gene silencing, and, optionally, a protein, antibody, or modulatory compound, if appropriate, and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration.

The use of such media and agents for pharmaceutically active substances is well known in the art. Except insofar as any conventional media or agent is incompatible with the active compound, use thereof in the compositions is contemplated. Supplementary active compounds can also be incorporated into the compositions.

### Exemplification

Throughout the examples, the following materials and methods were used unless otherwise stated.

Materials and Methods

In general, the practice of the present invention employs, unless otherwise indicated, conventional techniques of nucleic acid chemistry, recombinant DNA technology, molecular biology, biochemistry, and cell and cell extract preparation. See, e.g., DNA Cloning, Vols. 1 and 2, (D. N. Glover, Ed. 1985); Oligonucleotide Synthesis (M. J. Gait, Ed. 1984); Oxford Handbook of Nucleic Acid Structure, Neidle, Ed., Oxford Univ Press (1999); RNA Interference: The Nuts & Bolts of siRNA Technology, by D. Engelke, DNA Press, (2003); Gene Silencing by RNA Interference: Technology and Application, by M. Sohail, CRC Press (2004); Sambrook, Fritsch and Maniatis, Molecular Cloning: Cold Spring Harbor Laboratory Press (1989); and Current Protocols in Molecular Biology, eds. Ausubel et al., John Wiley & Sons (1992). See also PCT/US03/36551 (Attorney Docket No. UMY-04 1 PC); PCT/US03/24595 (Attorney Docket No. UMY-061PC); and PCT/US03/30480 (Attorney Docket No. UMY-062PC), of which all are incorporated in their entireties by reference herein.

siRNA Preparation

RNAs of the invention were chemically synthesized as 2′ bis(acetoxyethoxy)-methyl ether-protected oligos by Dharmacon (Lafayette, Colo.). Synthetic oligonucleotides were deprotected, annealed and purified as described by the manufacturer. Successful duplex formation was confirmed by 20% non-denaturing polyacrylamide gel electrophoresis (PAGE). All siRNAs were stored in DEPC (0.1% diethyl pyrocarbonate)-treated water at −80° C. The sequences of GFP or RFP target-specific siRNA duplexes were designed according to the manufacturer's recommendation and subjected to a BLAST search against the human genome sequence to ensure that no endogenous genes of the genome were targeted.

Culture and Transfection of Cells

HeLa cells were maintained at 37° C. in Dulbecco's modified Eagle's medium (DMEM, Invitrogen) supplemented with 10% fetal bovine serum (FBS), 100 units/ml penicillin and 100 pg/ml streptomycin (Invitrogen). Cells were regularly passaged at sub-confluence and plated 16 hr before transfection at 70% confluency. Lipofectamine (Invitrogen)-mediated transient cotransfections of reporter plasmids and siRNAs were performed in duplicate 6-well plates as described by the manufacturer for adherent cell lines. A transfection mixture containing 0.16-0.66 μg pEGFP-C1 and 0.33-1.33 μg pDsRed1-N1 reporter plasmids (Clontech), various amounts of siRNA(1.0 nM -200 nM), and 10 μl lipofectamine in 1 ml serum-reduced OPTI-MEM (Invitrogen) was added to each well. Cells were incubated in transfection mixture for 6 hours and further cultured in antibiotic-free DMEM. Cells were treated under same conditions without siRNA for mock experiments. At various time intervals, the transfected cells were washed twice with phosphate buffered saline (PBS, Invitrogen), flash frozen in liquid nitrogen, and stored at −80° C. for reporter gene assays.

In Vivo Fluorescence Analysis

pEGFP-C1, pDsRed1-N1 reporter plasmids and 50 nM siRNA were cotransfected into HeLa cells by lipofectamine as described above except that cells were cultured on 35 mm plates with glass bottoms (MatTek Corporation, Ashland Mass.) instead of standard 6-well plates. Fluorescence in living cells was visualized 48 hours post transfection by conventional fluorescence microscopy (Zeiss). For GFP and RFP fluorescence detection, FITC and CY3 filters were used, respectively.

Dual Fluorescence Efficacy Assay

The Dual Fluorescence Efficacy Assay was carried out essentially as described in PCT/US03/30480. Briefly, HeLa cells were maintained at 37° C. in Dulbecco's modified Eagle's medium (DMEM, Invitrogen) supplemented with 10% fetal bovine serum (FBS), 100 units/ml penicillin, and 100 μg/ml streptomycin (Invitrogen). Cells were regularly passaged at subconfluence and plated 16 hr before transfection at 70% confluency. Lipofectamine (Invitrogen)-mediated transient cotransfections of reporter plasmids and siRNAs were performed in duplicate 6-well plates. A transfection mixture containing 0.16 μg pEGFP-C1 and 0.33 μg pDsRed2-N1 reporter plasmids (Clontech), various amount of siRNA (From 0.5 nM to 400 nM), and 10 μl lipofectamine in 1 ml serum-reduced OPTI-MEM (Invitrogen) was added to each well. Cells were incubated in transfection mixture for 6 hr and further cultured in antibiotic-free DMEM. Cells were treated under the same conditions without siRNA for mock experiments. At various time intervals, the transfected cells were washed twice with phosphate-buffered saline (PBS, Invitrogen), flash frozen in liquid nitrogen, and stored at −80° C. for reporter gene assays.

Fluorescence of GFP in cell lysates was detected by exciting at 488 nm and recording from 498-650 or 504-514 nm. The spectrum peak at 507 or 509 nm represents the fluorescence intensity of GFP. Fluorescence of RFP2 in the same cell lysates was detected by exciting at 558 or 568 nm and recording from 578 to 588 nm or 588 to 650 nm. The spectrum peak at 583 nm represents the fluorescence intensity of RFP2. The fluorescence intensity ratio of target (EGFP) to control (RFP2) fluorophore was determined in the presence of siRNA duplex and normalized to that observed in the mocked treated cells. Normalized ratios less than 1.0 indicates specific interference.

Preparation of Cell Extracts

HeLa cell cytoplasmic extract was prepared following the Dignam protocol for isolation of HeLa cell nuclei (Dignam et al., 1983). The cytoplasmic fraction was dialysed against cytoplasmic extract buffer (20 mM Hepes, pH 7.9, 100 mM KCl, 200 μM EDTA, 500 μM DTT, 500 μM PMSF, 2 mM MgCl₂ 10% glycerol). The extract was stored frozen at −70° C. after quick-freezing in liquid nitrogen. The protein concentration of HeLa cytoplasmic extract varied between 4 to 5 mg/ml as determined by using a BioRad protein assay kit.

Preparation of Primed Mammalian Cells and Cell Extracts Having High RISC Activity

Cells were transfected with chemically synthesized single strand (sense or antisense) or duplex siRNAs (Dharmacon). After 24 h of transfection, cells were harvested to prepare cell extracts. Cytoplasm from HeLa cells was prepared following the Dignam protocol for isolation of HeLa cell nuclei (Dignam et al. 1983). The cytoplasmic fraction was dialyzed against cytoplsmic extract buffer (20 mM Hepes, pH 7.9, 100 mM KCl, 200 μM EDTA, 500 μM DTT, 500 μM PMSF, 2 mM MgCl₂, 10% glycerol). The extract can be stored frozen at −70° C. after quick-freezing in liquid nitrogen. The protein concentration of HeLa cytoplasmic extract varied between 4 to 5 mg/ml as determined by Biorad protein assay kit.

Preparation of Cap-Labeled Target mRNA

For mapping of the target RNA cleavage, a 124 nt EGFP transcript, corresponding to nts 195-297 relative to the start codon followed by the 21 nt complement of the SP6 promoter sequence, was amplified from template pEGFP-C1 by PCR using 5′ primer GCCTAATACGACTCACTATAGGACCTACGGCGTGCAGTGC (T7 promoter underlined) and 3′ primer TTGATTTAGGTGACACTATAGATGGTGCGCTCCTG-GACGT (SP6 promoter underlined). Alternatively, the GFP target sequence was amplified by PCR with forward and reverse primers 5′-GCCTAATACGACTCACTATAGACCTACGGCGTGCAGTGC-3′ and 5′-TTTTTTTTTTTTTTTTTTTTTTTTGATGGTGCGCTCCTGGACGT-3′, respectively, for transcription of a 126-nt GFP target RNA containing a 24-nt adenosine tail. The resulting transcripts were ³²P-cap-labeled, as previously described (Chiu et al., RNA 9:1034-48 (2003). His-tagged mammalian capping enzyme was expressed in E. coli and purified to homogeneity. Guanylyltransferase labeling was performed by incubating 1 nmole of transcripts with 50 pmole his-tagged mammalian capping enzyme in the 100 μl capping reaction containing 50 mM Tris-HCl (pH 8.0), 5 mM DTT, 2.5 mM MgCl₂, 1 U/μl RNasin RNase inhibitor (Promega) and [α-³²P]GTP at 37° C. for 1 h. Reactions were chased for 30 min by supplementing GTP concentration to 100 μM. Cap-labeled target mRNA were resolved on 10% polyacrylamide-7 M urea gel and purified.

In Vitro Target mRNA Cleavage Assay

siRNA-mediated cleavage of target mRNA in human cytoplasmic extract was performed as described (Martinez et al. 2002) with some modifications. siRNA duplexes were pre-incubated in HeLa cytoplasmic extract at 37° C. for 15 min prior to addition of the 124 nt cap-labeled target mRNA generated as described above. After addition of all components, final concentrations were 500 nM siRNA, 50 nM target mRNA, 1 mM ATP, 0.2 mM GTP, 1 U/μl RNasin, 30 μg/ml creatine kinase, 25 mM creatine phosphate, and 50% S100 extract. Incubation was continued for 1.5 h. Cleavage reactions were stopped by the addition of 8 volumes of proteinase K buffer (200 mM Tris-HCl [pH 7.5], 25 mM EDTA, 300 mM NaCl, and 2% w/v SDS). Proteinase K, dissolved in 50 mM Tris-HCl [pH 8.0], 5 mM CaCl₂, and 50% glycerol, was added to a final concentration of 0.6 mg/ml. Reaction products were extracted with phenol/chloroform/isoamyl alcohol (25:24:1), chloroform and precipitated with 3 volumes of ethanol. Samples were separated on 8% polyacrylamide-7 M Urea gels.

### EXAMPLE 1

### Separate and Temporal Administration of RNAi/gene Silencing Agents are Effective and Bypass an Interferon Response

The following example describes methods for conducting RNAi/gene silencing without activating an interferon response pathway, by the separate and temporal administration of each single-strand of a double-stranded siRNA agent.

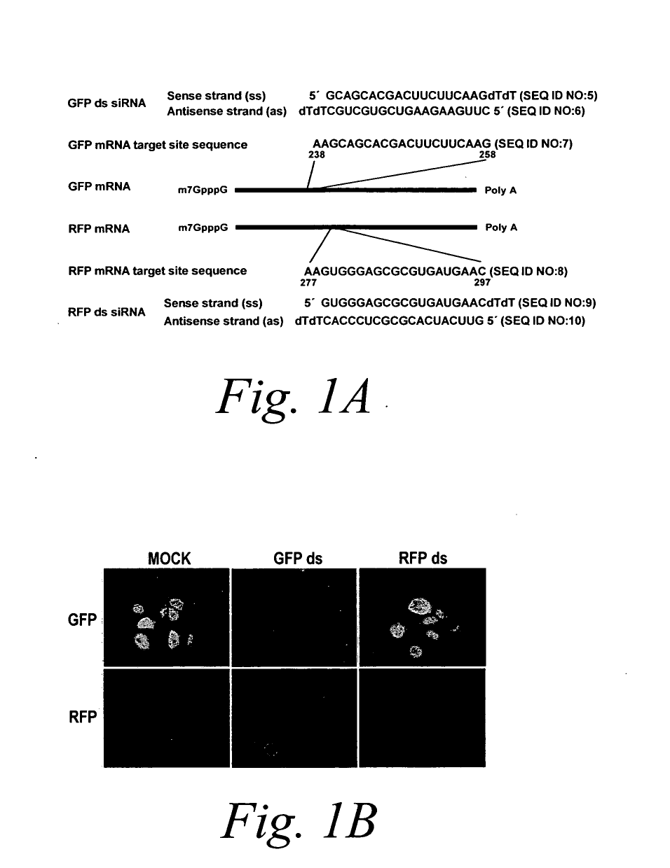

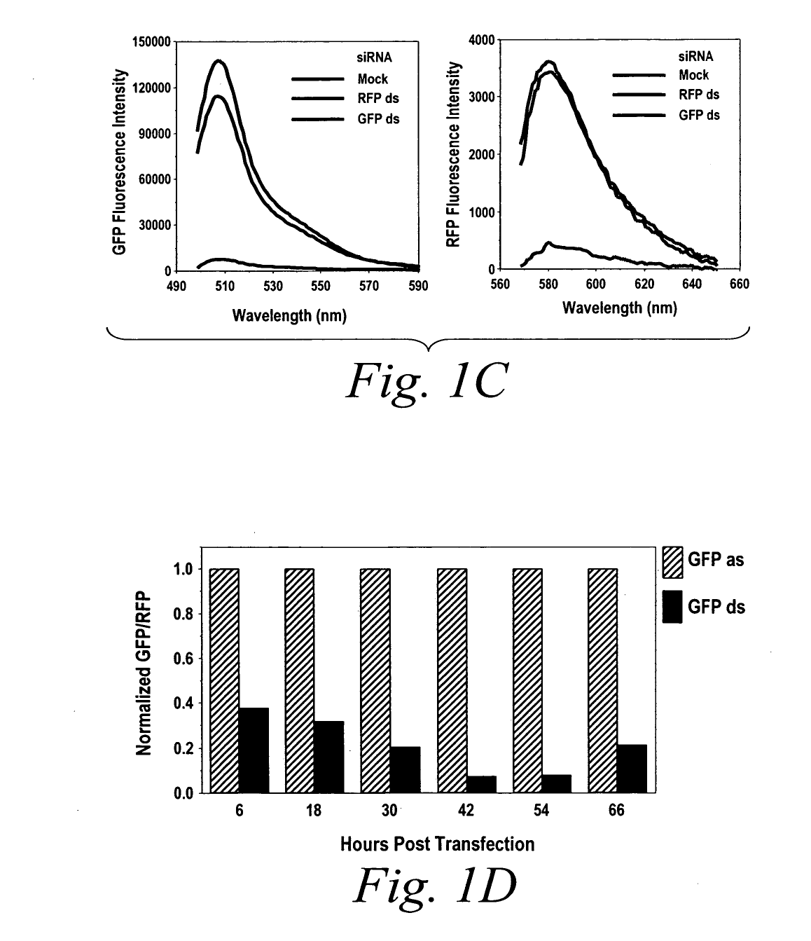

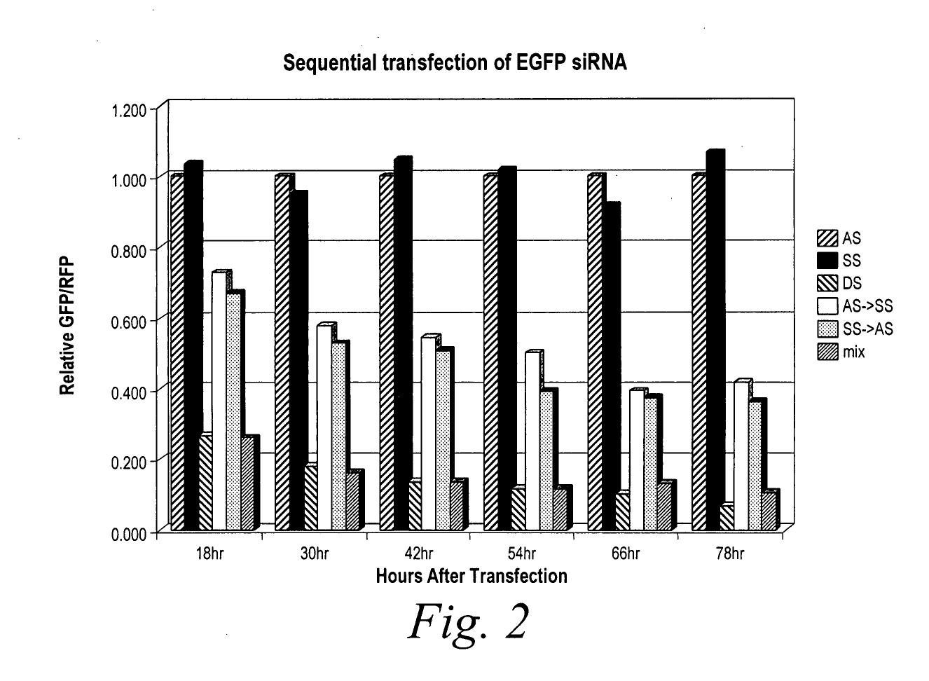

To determine the efficacy of sequential administration of each strand of a double stranded siRNA agent, the efficacy assay described above was used which indicates the amount of RNAi/gene silencing as a function of suppressed fluorescence as compared to an internal control (see FIG. 1). Cells were transfected as described above with either an antisense strand (AS), sense strand (SS), annealed antisense and sense duplex (DS), non-annealed antisense and sense duplex (mix), or first with the antisense strand and 3 to 6 hours later with the sense strand (AS>SS) or the reverse order (SS>AS) and the amount of suppressed fluorescence was measured (see FIG. 2). Surprisingly, non-annealed strands (mix) were equally effective as the annealed strands (DS) and the separate administration of each strand over a 3 to 6 hours was substantially effective in gene silencing.

Accordingly, these results indicate that double stranded siRNA agents can be administered separately as single-strands and over a period of time and still be substantially effective in RNAi/gene silencing of given target gene.

### EXAMPLE 2

### Non-Canonical RNAi/gene Silencing Agents are Effective and Bypass Length and Overhang Requirements

The following example describes methods for conducting effective RNAi/gene silencing using non-canonical siRNA agents which bypass length and overhang requirements.

To determine the efficacy of the non-canonical siRNAs, the efficacy assay described above was used which indicates the amount of RNAi/gene silencing as a function of suppressed fluorescence as compared to an internal control (see FIG. 1). To determine siRNA length and overhang requirements, a panel of siRNAs having non-canonical length and/or overhangs was synthesized (see FIG. 3). As shown in FIG. 3, a canonical siRNA with 19 nucleotides of complementarity and having 5′ and 3′ dTdT canonical overhangs was tested along side selected test siRNAs having deletions of 1, 2, or 3 nucleotides and lacking at least one canonical overhang (see canonical siRNA labeled 1 and non-canonical test siRNAs labeled 2, 5, 8, and 11 of FIG. 3).

Surprisingly, the non-canonical siRNAs where all effective in gene silencing (suppressed fluorescence) whether annealed or non-annealed. In particular, the test siRNA having a non-canonical overhang and having 3 nucleotide deletions was as effective as the canonical siRNA.

Accordingly, these results indicate that non-canonical siRNA agents which bypass length and overhang requirements are effective siRNA agents.

### EXAMPLE 3

### Modified siRNAs Reveal Stoichiometry of RNAi/gene Silencing Machinery

The following example describes methods for determining the amount of RISC present in a cell using modified siRNA agents.

Understanding the consequences of complex RNAi activities in mammalian cells is highly desirable. Accordingly, the invention also provides modified siRNA agents which can reveal the stoichiometry of RNAi/gene silencing machinery. In particular, by administering a titration of double-stranded siRNA nucleic acids having one or more nucleotide modifications, e.g., 2′-O-methylation, against an unmodified siRNA, a calculation of per cell amounts of RNAi activity, e.g., RISC activity, was determined.

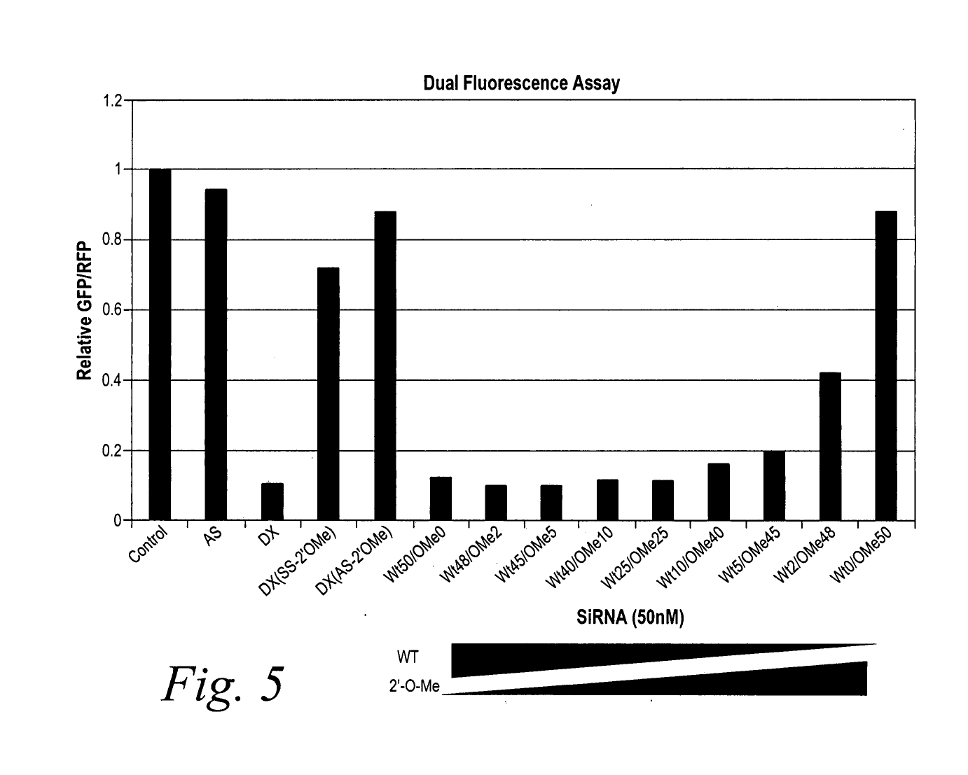

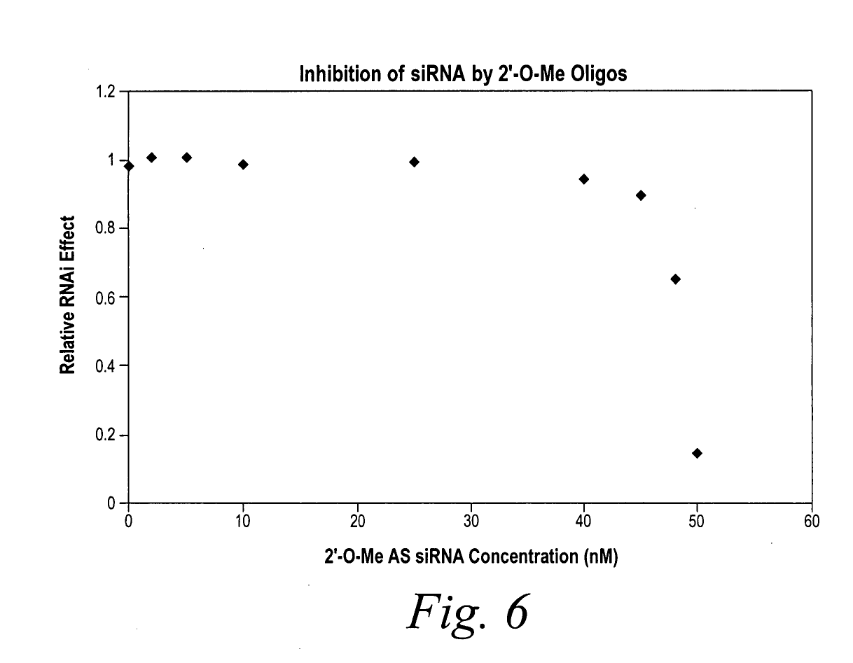

Cells were transfected as described above with an siRNA agent wherein a percentage of each strand has been modified with a 2′O-methyl group at each nucleotide base. As shown in FIG. 5, when the test siRNA comprises increasing amounts of methylated siRNA, the amount of gene silencing is increasingly reduced to baseline, i.e., where no gene silencing has occurred. These data are further represented in FIG. 6 as the lack of RNAi as a function of increasing amounts of 2′-O-methylated siRNA concentration. Still further, these data allowed for the calculation of a pre cell concentration of RISC activity as between about 0.2 to 1.9 nM.

Accordingly, these results indicate that modified siRNAs can be successfully titrated into the RISC complex and reveal the stoichiometry of such RNAi machinery. In addition, these results show that concentrations of siRNA can be reduced from 50 nM to a range of about 1-5 nM or less, especially, e.g., if the cells, cell extracts, or organisms are first primed.

### EXAMPLE 4

### Non-Canonical siRNAs are Suitable for Inducing RNAi In Vivo

The following example describes methods for conducting effective RNAi/gene silencing in vivo using non-canonical siRNA agents.

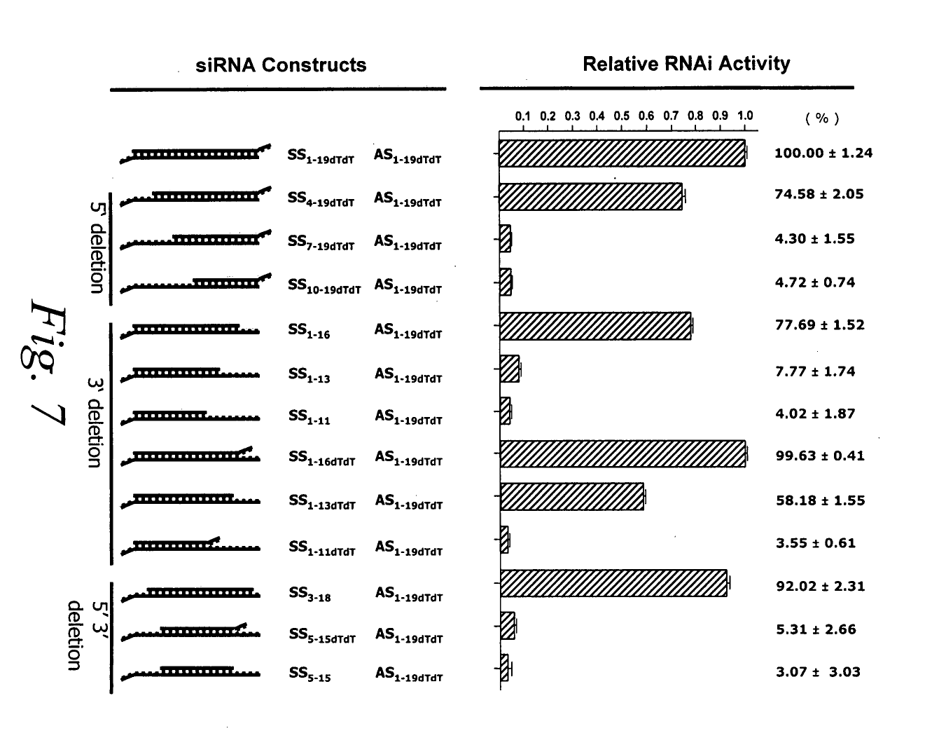

Briefly, to delineate the minimum dsRNA A-form helical structure required to assemble catalytically active RISC (also referred to herein as RISC*), siRNA duplexes were designed targeting Green Fluorescent Protein (GFP) that have an antisense strand of 19-nts plus dTdT (19-nt dTdT) and a sense strand harboring deletions at the 5′- or 3 ′-ends (FIG. 7). RNAi activity of these siRNA duplexes was quantitatively analyzed in a dual fluorescence reporter system as described previously. Amounts of 50 nM of wild type 19-nt dTdT siRNA showed 92% silencing of GFP expression in HeLa cells 48 h post-transfection and this activity was denoted as 100% in FIG. 7 for comparison with other siRNA sequences. Analysis of 5′ deletions showed that a 16-nt plus dTdT (16-nt dTdT) sense strand (SS_{4-19dTdT}) induced RNAi with ˜75% efficiency while two other deletions, SS_{7-19dTdT} and SS_{10-19dTdT}, did not exhibit RNAi activity. To map the 3′-end boundary required for siRNA function, the 3′ end of the sense-strand was systemically deleted. A sense strand containing 16 nts (SS₁₋₁₆) showed efficient RNAi (˜77%). Duplexes shorter than 16 nts (SS₁₋₁₃ and SS₁₋₁₁) were inactive. Because 19-nt siRNA duplexes with dTdT overhangs have improved RNAi efficiency, the effect of adding dTdT to the truncated 3′-end of the sense strand by quantifying the level of GFP knocked down by duplexes SS_{1-16dTdT} and SS_{1-11dTdT} was determined (FIG. 7). In particular, SS_{1-16dTdT} exhibited enhanced RNAi function that was comparable to wild-type 19-nt dTdT siRNA. Addition of dTdT to SS₁₋₁₁ did not increase the RNAi efficiency of the 11-nt duplex.

To address which region of the siRNA has to form a duplex structure to cause RNAi, sense strands with deletions at both the 5′ and 3′ ends were synthesized and tested for their RNAi function. The 16-nt SS₃₋₁₈ showed high efficiency GFP knockdown (˜92%), however, 11-nt SS₅₋₁₅ was non-functional and addition of dTdT did not improve the RNAi function of the shortened duplex (FIG. 7). These findings demonstrate that efficient RNAi can be accomplished using a 19-nt dTdT antisense strand and a 16-nt sense strand. Taken together, these results indicate that a 16-nt duplex RNA structure is suitable for gene silencing in vivo.

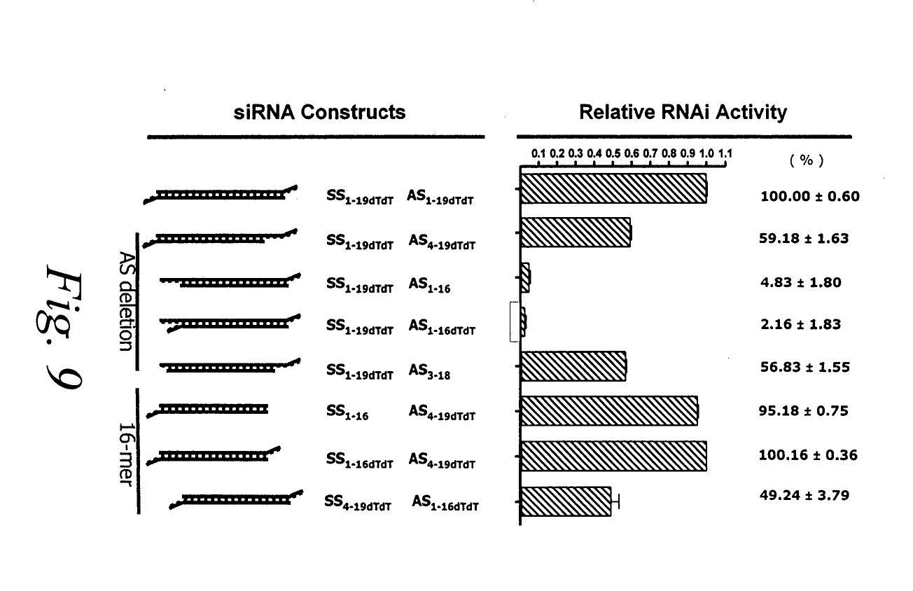

Reciprocal experiments were performed to ascertain whether this 16-nt rule applied to the antisense strand. In these experiments, dsRNA duplexes harbored a 19-nt dTdT sense strand and an antisense strand truncated from the 5′ and/or 3′ ends (FIG. 9). AS_{4-19dTdT} exhibited decreased RNAi efficiency (˜59%), but the 5-nt sense strand overhang created upon deleting antisense nts 1-3 contributed to the loss in function since increases in 3′ overhang length have been determined to have detrimental effects on RNAi. The AS₃₋₁₈ showed intermediate RNAi activity (˜57%) and as above, is due to the 4-nt sense strand overhang which contributed to the loss of function. These results indicate that a 19-nt dTdT sense strand and a 16-nt or 16-nt dTdT antisense strand can induce RNAi. Surprisingly, AS₁₋₁₆ and AS_{1-16dTdT} did not exhibit RNAi activity, indicating that nts 17-19 of the antisense strand can be important for target RNA recognition.

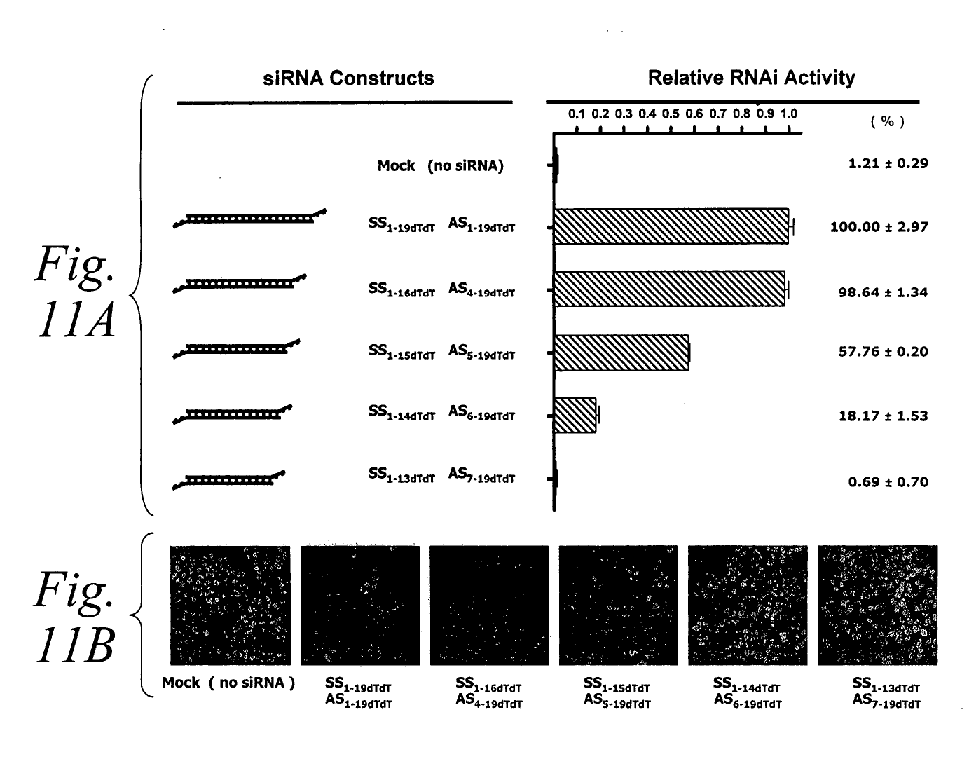

The 16-nt rule was also tested to determine if it applied to duplexes in which both strands were truncated (FIG. 11). The 16-nt dTdT siRNA induced RNAi at a high efficiency (˜99%), the 15-nt dTdT siRNA induced knockdown at a moderate efficiency (˜58%) whereas 14-nt dTdT and 13-nt dTdT siRNAs induced knockdown at low efficiencies (˜18% and ˜1%, respectively). Collectively, these results demonstrate that 16-nt dTdT is the threshold number of nucleotides required for inducing highly efficient gene knockdown.

### EXAMPLE 5

### Non-Canonical siRNAs-Programmed RISC Cleave Target RNAs In Vitro

The following example describes methods for conducting effective RNAi/gene silencing in vivo using non-canonical siRNA agents to program RISC activity.

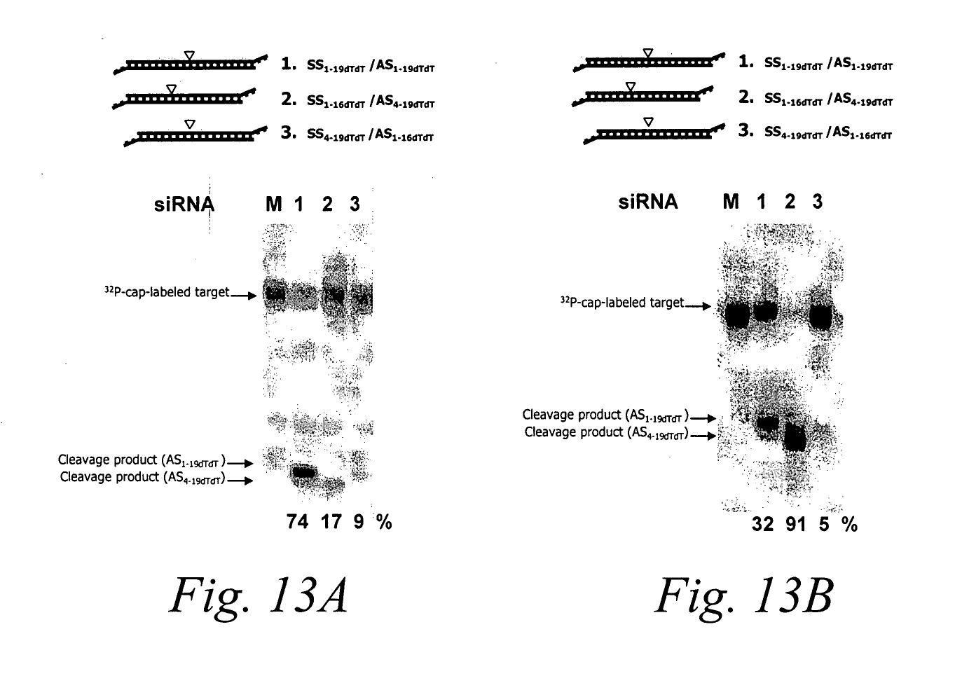

Briefly, SS_{1-16dTdT} AS_{4-19dTdT} exhibited wild-type levels of GFP knockdown (FIG. 9), indicating that a 16-nt dTdT siRNA is as efficient at causing RNAi in vivo as a 19-nt dTdT siRNA. To show that 16-nt dTdT siRNA entered the RNAi pathway, the RNAi efficiency of target mRNA cleavage in vitro when RISC was primed with 16-nt dTdT siRNA was measured. HeLa cells were transfected with 19-nt dTdT or 16-nt dTdT siRNA to program RISC. Cell extracts from transfected cells were then incubated with a 126-nt ³²P-cap-labeled GFP mRNA target to measure the activity of activated siRNA-programmed RISC (siRISC*). The 19-nt dTdT siRISC* and two different 16-nt dTdT siRISC* enzymes were shown to cleave the target RNA (FIG. 13A. ˜74%, ˜17%, and ˜9%, cleavage, respectively), indicating that 16-nt dTdT siRNA enters the RNAi pathway. Interestingly, the cleavage product of SS_{1-16dTdT} AS_{4-19dTdT} siRISC* revealed that the cleavage site had shifted 3 nts (FIG. 13A; compare lanes 1 and 2 and see arrows), reflecting the new position of the 5′ end of the antisense strand after truncating 3 nts.

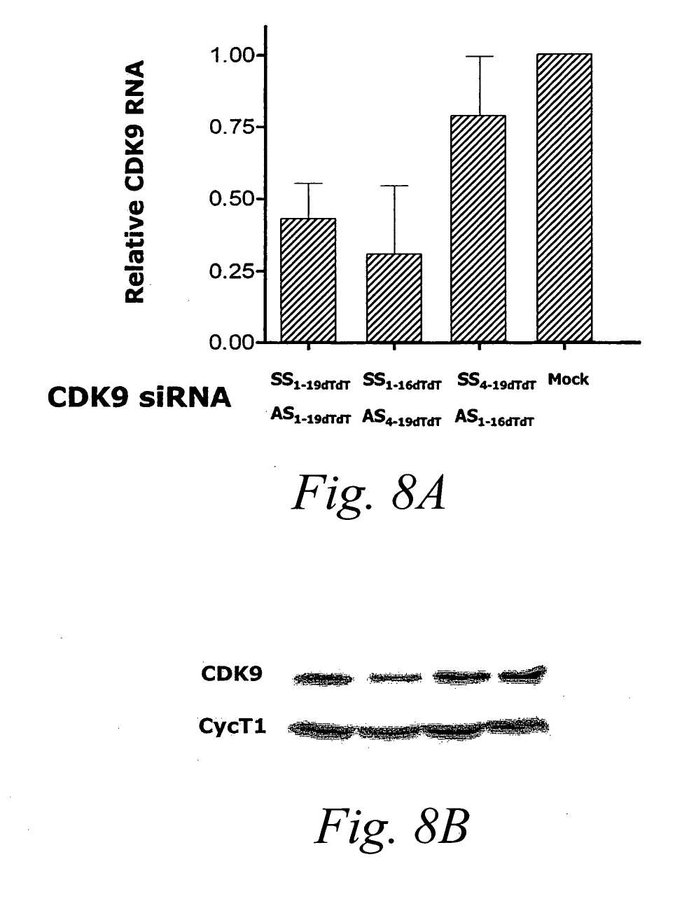

The RNAi-inducing capacity of 16-nt dTdT and 19-nt dTdT siRNA was also compared for their ability to target the transcription elongation factor CDK9 RNA. The 16-nt dTdT (SS_{1-16dTdT} AS_{4-19dTdT}) and 19-nt dTdT (SS_{1-19dTdT} AS_{4-19dTdT}) CDK9 siRNA knocks down CDK9 expression levels in vivo with an efficiency of ˜70% and ˜57%, respectively (FIG. 8). The efficiency of 16-nt dTdT (SS_{4-19dTdT} AS_{1-16dTdT}) siRNA showed a lower efficiency of knockdown at 25% (FIG. 8). In vitro cleavage activity was measured by incubating CDK9 siRNA-programmed HeLa extract with a 150-nt ³²P-cap-labeled CDK9 substrate RNA. The 19-nt dTdT (SS_{1-19dTdT} AS_{4-19dTdT}) siRISC* showed ˜32% cleavage while the 16-nt dTdT (SS_{1-16dTdT} AS_{4-19dTdT}) siRISC* showed ·91% cleavage (FIG. 13B, lanes 1 and 2), indicating that siRISC* activity induced by the 16-nt dTdT siRNA was robust. The 16-nt dTdT (SS_{4-19dTdT} AS_{1-16dTdT}) siRNA showed ˜5% cleavage (FIG. 13B, lane 3), reflecting the lower RNAi efficiency observed in vivo. The cleavage product resulting from CDK9 16-nt dTdT (SS_{1-16dTdT} AS_{4-19dTdT}) siRISC* activity reflected a 3-nt shift in the cleavage site (FIG. 13B, compare lanes 1 and 2 and see arrows) that was similar to the shift seen with GFP 16-nt dTdT (SS_{1-16dTdT} AS_{4-19dTdT}) siRNA.

These results demonstrate that the 5′ end of the truncated antisense guide strand defined a new cleavage site in the mRNA target ˜10-11 nt upstream of nt 4 of the antisense strand, which is consistent with the guide rule documented for 19-nt dTdT siRNA. Taken together with the potent cleavage activity of CDK9 16-nt dTdT siRISC*, these results indicate that 16-nt dTdT siRNA is a bona fide inducer of RISC-mediated gene silencing.

### EXAMPLE 6

### Non-Canonical RNAi-Inducing Capacity of 16-nt dTdT and 19-nt dTdT siRNA In Vivo

The following example describes methods for identifying the RNAi-inducing capacity of non-canonical siRNA agents in vivo.

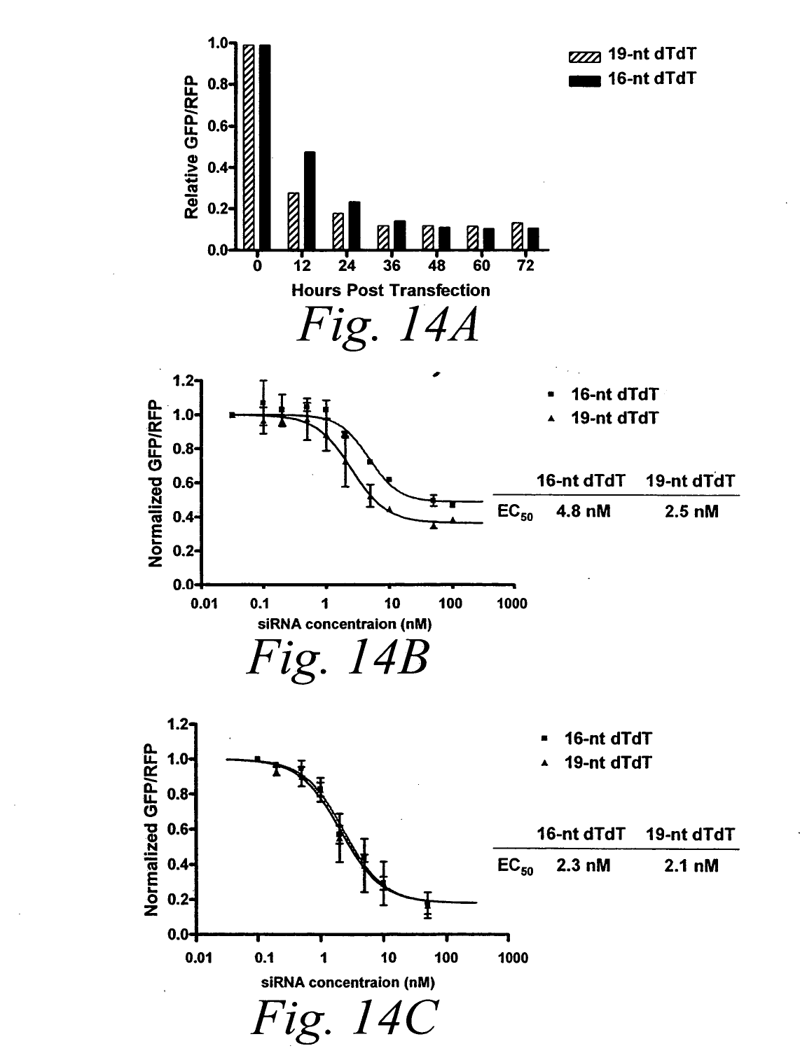

Briefly, the difference in the GFP 16-nt dTdT and 19-nt dTdT siRISC cleavage efficiencies in vitro was further explored to determine the RNA-inducing capacity of these siRNAs in vivo. A time-course experiment was performed to determine when the knockdown caused by the GFP 16-nt dTdT and 19-nt dTdT siRNAs peaked. A 50 nM amount of the GFP 16-nt dTdT (SS_{1-16dTdT} AS_{4-19dTdT} was used for this and all subsequent experiments) or 19-nt dTdT siRNA was transfected into cells and the knockdown efficiency of both siRNAs was measured at 12 h intervals for 72 h. By 12 h post-transfection, GFP levels were knocked down ˜72% and ˜52% by the 19-nt dTdT and 16-nt dTdT siRNA, respectively (FIG. 14A). By 36 h, however, the 16-nt dTdT and 19-nt dTdT siRNAs showed similar knockdown efficiencies (FIG. 14A; ˜85% and ˜88%, respectively). These results show that the 16-nt dTdT siRNA may initially induce RNAi effects at a slower rate than the 19-nt dTdT siRNA but the capacity of the 16-nt dTdT and 19-nt dTdT siRNAs to knockdown GFP levels becomes similar overtime.

The EC₅₀ of the GFP 16-nt dTdT and 19-nt dTdT siRNAs was also determined for different time points by transfecting increasing concentrations of the siRNAs (0.01-200 nM) into HeLa cells. The level of GFP knockdown was then measured 12 h and 60 h post-transfection. The EC₅₀ of the 16-nt dTdT and 19-nt dTdT siRNAs was 4.8 nM and 2.5 nM, respectively, 12 h post-transfection (FIG. 5B) and 2.3 nM and 2.1 nM, respectively, 60 h post-transfection (FIG. 14C). These results indicate a higher concentration of GFP 16-nt dTdT siRNA than that of 19-nt dTdT siRNA was required to initially induce RNAi. However, as observed during the knockdown time course above, the capacity of 16-nt dTdT siRNA to knockdown GFP levels becomes equivalent to that of 19-nt dTdT siRNA over time.

### EXAMPLE 7

### Non-Canonical siRNA 16-nt dTdT AND 19-nt dTdT siRISC Activity Cleave Target RNAs with Different Efficiencies

The following example describes methods for identifying the differential RNAi-inducing capacity of non-canonical siRNA agents in vivo.

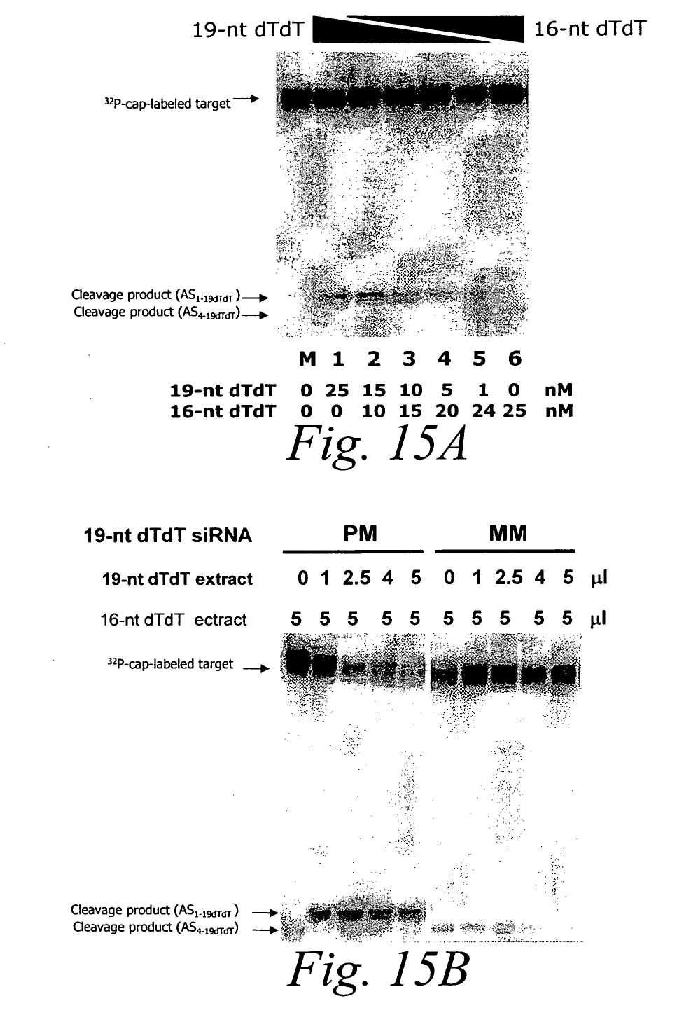

To determine whether the GFP 16-nt dTdT siRISC* can effectively compete with 19-nt dTdT siRISC* for target RNA, HeLa cells were transfected with both 16-nt dTdT and 19-nt dTdT siRNA to program RISC. The concentration of each siRNA varied from 0-25 nM. In vitro cleavage assays were then performed with the prepared extracts and GFP target RNA. The 19-nt dTdT and 16-nt dTdT siRISC* activity in the same extract was distinguished by the size of the cleavage products, which differed because the 5′ end of the 16-nt dTdT was truncated by 3 nts. Interestingly, the cleavage product resulting from 19-nt dTdT siRISC* predominated even when the concentration of the 16-nt dTdT siRNA was titrated to 20 nM and the 19-nt dTdT to 5 nM (FIG. 15, lanes 1-4). The 16-nt dTdT siRISC* cleavage product became apparent only when the concentration of the 16-nt dTdT siRNA was 24 nM and that of the 19-nt dTdT siRNA was 1 nM but even at this concentration ratio, the 19-nt dTdT siRISC* cleavage product was still clearly observed (FIG. 15, lane 5). The equal amount of cleavage products resulting at this 24 to 1 concentration ratio siRNA indicated that 19-nt dTdT siRISC* has a higher binding affinity for target mRNA then 16-nt dTdT siRISC*.

To distinguish whether GFP 16-nt dTdT siRISC* and 19-nt dTdT siRISC* could effectively compete for the same target RNA during catalysis, in vitro cleavage assays were performed after mixing different amounts of 16-nt dTdT-primed extracts and 19-nt dTdT-primed extracts (FIG. 15). Even when using the highest amount of 16-nt dTdT extract and lowest amount of 19-nt dTdT extract, the cleavage product of 19-nt dTdT siRISC* predominated, indicating that 19-nt dTdT siRISC* was more effective catalytically than 16-nt dTdT siRISC*. As a control, different amounts of 16-nt dTdT extract were mixed with extracts programmed with a non-functional GFP 19-nt dTdT siRNA (mismatch or mm) that was mismatched at nucleotides normally complementary to the target cleavage site. The cleavage product of the 16-nt dTdT siRISC* was the only product observed but the cleavage efficiency was reduced, indicating that mismatched 19-nt dTdT siRISC* also competed with 16-nt dTdT siRISC* for target RNA.

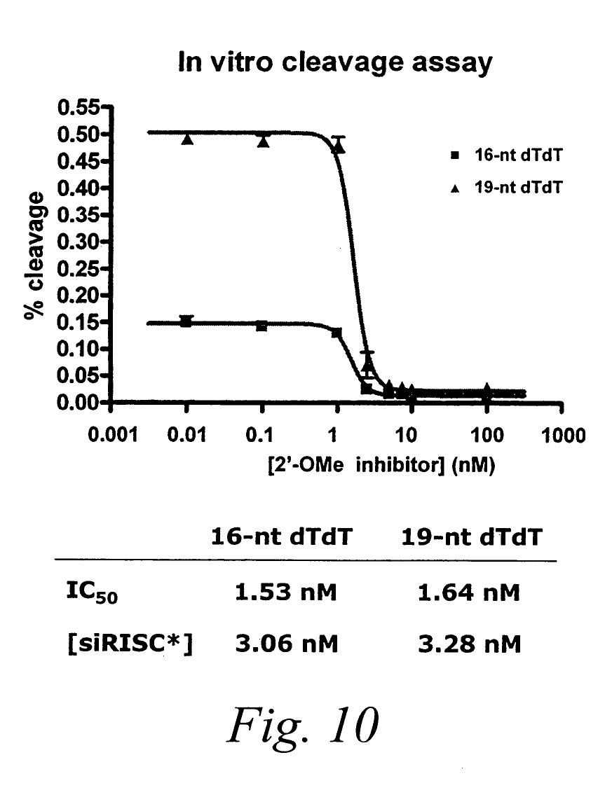

The concentration of GFP siRISC* in the 16-nt dTdT and 19-nt dTdT extracts was determined by using a method in which a 2′-O-methyl oligonucleotide complementary to the antisense siRNA strand complexed with siRISC* blocks the activity of GFP siRISC* (Hutvagner et al. PLoS Biol 2:E98 (2004). The amount of 16-nt dTdT and 19-nt dTdT siRISC* programmed in HeLa cells at increasing concentrations of siRNA was saturated to yield ˜3.06 nM and 3.28 nM siRISC*, respectively (FIG. 10), indicating that the 16-nt dTdT and 19-nt dTdT siRNAs programmed similar amounts of siRISC*. Taken together with their differing cleavage efficiencies, these results indicate that GFP 16-nt dTdT siRISC* cleaves target RNA less efficiently than 19-nt dTdT siRISC*.

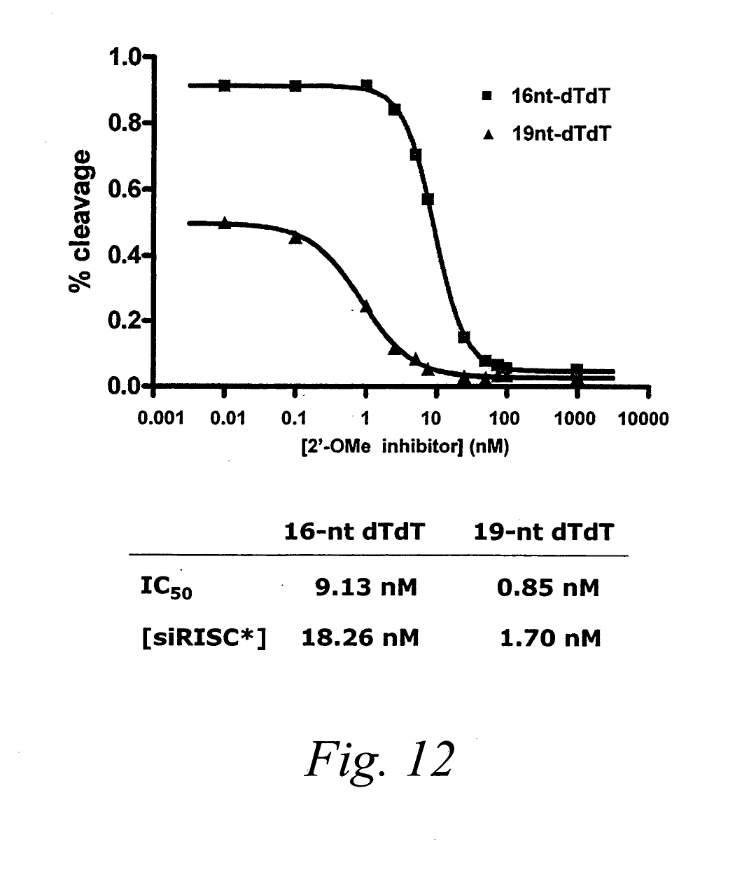

Because CDK9 16-nt dTdT siRISC* cleaved target RNA with such a high efficiency, the concentration of CDK9 16-nt dTdT and 19-nt dTdT siRISC* was also determined. Remarkably, the amount of 16-nt dTdT and 19-nt dTdT siRISC* programmed in HeLa cells at increasing concentrations of siRNA was saturated to yield 18.26 nM and 1.70 nM siRISC*, respectively (FIG. 12), indicating that the 16-nt dTdT siRNA programmed ˜10× more siRISC* than 19-nt dTdT siRNA. These results indicate that CDK9 16-nt dTdT siRISC* cleaves target RNA with much greater efficiency than 19-nt dTdT siRISC* because 16-nt dTdT siRNA has the capacity to program a greater concentration of RISC. These findings indicate that RNAi potency can correspond to the amount of siRISC* formed by a given siRNA and that the length of siRNA can dictate how much siRISC* is formed.

### Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A small interfering RNA (siRNA), comprising a sense strand and an antisense strand, the antisense strand having a sequence sufficiently complementary to a target gene sequence to direct target-specific RNA interference (RNAi), wherein the strands, when aligned, form at least one non-canonical end.

2. The siRNA of claim 1, wherein the siRNA is selected from the group consisting of
an siRNA having a first non-canonical end,
an siRNA having a second non-canonical end,
an siRNA having a first and a second non-canonical end,
an siRNA wherein the sense strand can be shortened or truncated at the 5′ end and aligned such that its 3′ end overhangs the 5′ end of the antisense strand,
an siRNA wherein the sense strand can be shortened or truncated at the 3′ end and aligned such that the 3′ end of the antisense strand overhangs its 5′ end,
an siRNA wherein the sense strand can be shortened or truncated at the 3′ end, the 3′ end further comprising 2-3 non-complementary nucleotides, the sense strand being aligned such that the 3′ end of the antisense strand overhangs its 5′ end, and
an siRNA wherein the sense strand can be shortened or truncated at both ends, the 3′ end, optionally, further comprising 2-3 non-complementary nucleotides.

3. A small interfering RNA (siRNA), comprising a sense strand and an antisense strand, the antisense strand having a sequence sufficiently complementary to a target gene sequence to direct target-specific RNA interference (RNAi), wherein the sense strand and antisense strand are separately and temporally exposed to the target gene sequence.

4. The siRNA of claim 1 or 3, wherein the sense strand is about 19, 20, or 21 nucleotides and the corresponding antisense strand is at least 10, 11, 12, 13, 14, 15, 16, 17, 18, or 19 nucleotides.

5. The siRNA of claim 1 or 3, wherein the antisense strand is about 19, 20, or 21 nucleotides and the corresponding sense strand is at least 10, 11, 12, 13, 14, 15, 16, 17, 18, or 19 nucleotides.

6. The siRNA of claim 3, wherein the siRNA directs target specific interference and bypasses an interferon response pathway.

7. The siRNA of claim 1 or 3, wherein the strands are separately and temporally exposed to the target gene sequence over a time interval of about 1 hour or more.

8. The siRNA of claim 7, wherein the time interval is between about 1 hour to about 24 hours.

9. The siRNA of claim 7, wherein the time interval is between about 1 hour to about 48 hours.

10. The siRNA of claim 7, wherein the time interval is between about 1 hour to about 72 hours.

11. A composition comprising the siRNA molecule of any the preceding claims and a pharmaceutically acceptable carrier.

12. A vector encoding the siRNA molecule of claims 1 or 3.

13. The vector of claim 12, wherein the siRNA is capable of conditional expression.

14. The vector of claim 13, wherein conditional expression is achieved by a tet operator and operon.

15. A cell comprising the vector of claim 12, 13, or 14.

16. The cell of claim 15, wherein the vector is chromosomally integrated.

17. An organism comprising the cell of claim 15 or 16.

18. A method of activating target-specific RNA interference (RNAi) in a cell comprising,
introducing into the cell a small interfering RNA (siRNA), comprising a sense strand and an antisense strand, the antisense strand having a sequence sufficiently complementary to a target gene sequence to direct target-specific RNA interference (RNAi), wherein the strands, when aligned, form at least one non-canonical end,
the siRNA being introduced in an amount sufficient for degradation of target mRNA to occur, thereby activating target-specific RNAi in the cell.

19. The method of claim 18, wherein the sense and antisense strand are introduced separately.

20. The method of claim 19, wherein the sense and antisense strand are introduced separately and over a time interval of about 1 hour or more.

21. The method of claim 18, wherein the siRNA is introduced into the cell by contacting the cell with the siRNA.

22. The method of claim 21, wherein the siRNA is introduced into the cell by contacting the cell with a composition comprising the siRNA and a lipophilic carrier.

23. The method of claim 18, wherein the siRNA is introduced into the cell by transfecting or infecting the cell with a vector comprising nucleic acid sequences capable of producing the siRNA when transcribed in the cell.

24. The method of claim 18, wherein the siRNA is introduced into the cell by injecting into the cell a vector comprising nucleic acid sequences capable of producing the siRNA when transcribed in the cell.

25. The method of claim 24, wherein the vector comprises transgene nucleic acid sequences.

26. The method of any one of claims 18-25, wherein the target mRNA specifies the amino acid sequence of a protein involved or predicted to be involved in a human disease or disorder.

27. A cell obtained by the method of any one of claims 18-25.

28. The cell of claim 27, wherein the cell is of mammalian origin.

29. The cell of claim 28, wherein the cell is of human origin.

30. An organism derived from the cell of claim 27.

31. A method of activating target-specific RNA interference (RNAi) in an organism comprising,
administering to the organism the siRNA of any one of claims 1-10, the siRNA being administered in an amount sufficient for degradation of the target mRNA to occur, thereby activating target-specific RNAi in the organism.

32. The method of claim 31, wherein the target mRNA specifies the amino acid sequence of a protein involved or predicted to be involved in a human disease or disorder.

33. An organism obtained by the method of claim 31.

34. The organism of claim 33, wherein the organism is of mammalian origin.

35. The organism of claim 33, wherein the organism is of human origin.

36. The organism of any one of claims 33-35, wherein the target mRNA specifies the amino acid sequence of a protein involved or predicted to be involved in a human disease or disorder.

37. A method of treating a disease or disorder associated with the activity of a protein specified by a target mRNA in a subject comprising,
administering to the subject the siRNA of any one of claims 1-10, the siRNA being administered in an amount sufficient for degradation of the target mRNA to occur, thereby treating the disease or disorder associated with the protein.

38. A method for deriving information about the function of a gene in a cell or organism comprising,
introducing into the cell or organism the siRNA of any one of claims 1-10, and
maintaining the cell or organism under conditions such that target-specific RNAi can occur,
determining a characteristic or property of the cell or organism, and
comparing the characteristic or property to a suitable control, the comparison yielding information about the function of the gene.

39. A method of validating a candidate protein as a suitable target for drug discovery comprising,
introducing into a cell or organism the siRNA of any one of the preceding claims, and
maintaining the cell or organism under conditions such that target-specific RNAi can occur,
determining a characteristic or property of the cell or organism, and
comparing the characteristic or property to a suitable control,
the comparison yielding information about whether the candidate protein is a suitable target for drug discovery.

40. A kit comprising reagents for activating target-specific RNA interference (RNAi) in a cell or organism, the kit comprising:
the siRNA molecule of any one of the preceding claims, and
instructions for use.
